(19) United States
(12) Patent Application Publication
(10) Pub. No.: US 2022/0125770 A1
(43) Pub. Date: Apr. 28, 2022

(54) COMBINATION THERAPY OF ALK-POSITIVE NEOPLASIA

(71) Applicant: Academisch Medisch Centrum, Amsterdam (NL)

(72) Inventors: Rogier VERSTEEG, Amsterdam (NL); Mohamed HAMDI, Amsterdam (NL); Ellen Marion WESTERHOUT, Amsterdam (NL)

(21) Appl. No.: 17/442,925

(22) PCT Filed: Mar. 27, 2020
(86) PCT No.: PCT/EP2020/058736

§ 371 Date: Sep. 24, 2021

(30) Foreign Application Priority Data
Mar. 27, 2019 (EP) 19165603.2

(51) Int. Cl. A61K 31/439; A61P 35/00; A61K 31/5377

(52) U.S. Cl. CPC A61K 31/439; A61K 31/5377; A61P 35/00

(57) ABSTRACT

The invention relates to a pharmaceutical product comprising therapeutically effective amounts of:
(a) an ALK inhibitor or an antibody-drug conjugate directed to the ALK receptor, and
(b) a TNF-related apoptosis-inducing ligand (TRAIL) receptor agonist.

### FIELD OF THE INVENTION

This invention relates to compounds suitable for use in a combination therapy against ALK positive neoplasia, in particular neuroblastoma. In particular, the invention relates to pharmaceutical products comprising an ALK inhibitor or an antibody-drug conjugate directed to the ALK receptor and a TNF-related apoptosis-inducing ligand (TRAIL) receptor agonist.

### BACKGROUND

Neuroblastoma is an aggressive childhood tumor. High stage neuroblastoma usually respond to therapy by complete clinical remission, but most tumors relapse within a few years as therapy-resistant lethal disease. The reason why tumors initially go in complete remission, but relapse later as therapy-resistant disease is unsolved. The inventors have recently published that most neuroblastoma tumors consist of two tumor cell types⁵. Neuroblastoma includes lineage-committed adrenergic (ADRN) and precursor-like mesenchymal (MES) tumour cells, which can transdifferentiate into one another^{5,6}. ADRN and MES cell types share the same genetic defects, but are phenotypically divergent and express different genes. The predominant type is ADRN and displays differentiation markers of the adrenergic lineage. A minority of cells has a mesenchymal phenotype. The MES-cells resemble developmental precursor-type cells. MES-type neuroblastoma cells are in vitro more resistant to chemotherapy than ADRN-type cells and accumulate in post-therapy tumours⁵, suggesting that MES cells survive therapy and may seed relapses. This would imply that in order to prevent lethal relapses in neuroblastoma, both the ADRN-type and MES-type cells in the tumour have to be killed.

ALK is the only mutated gene in neuroblastoma that can be targeted by small molecules, which has prompted clinical trials with ALK inhibitors.

Activating ALK mutations are found in 10% of neuroblastoma⁸⁻¹¹ and are the only tumour-driving mutations that can be targeted by small molecule inhibitors. ALK inhibitors efficiently kill neuroblastoma cell lines with ALK mutations in vitro and induce tumour regression in xenograft models of these cell lines¹². Recently, it has been demonstrated that even in neuroblastoma wherein ALK is unmutated, targeting the ALK pathway is effective. Antibody-drug conjugates directed to the ALK receptor have shown efficacy in preclinical models of neuroblastoma (Sano et al., Science Translational Medicine 13 Mar. 2019) Vol. 11, Issue 483).

Notwithstanding, many neuroblastoma relapse after treatment. It is therefore an objective of the invention to provide a treatment which is more effective, i.e. is capable of preventing a relapse or which is effective in case a relapse has occurred.

### SUMMARY OF THE INVENTION

The present invention is based on the surprising finding that MES-type neuroblastoma cells are resistant to ALK inhibition, but sensitive to killing by TRAIL, while ADRN-type neuroblastoma cells are resistant to TRAIL but killed by ALK inhibition. Consequently, treatment of neuroblastoma by a combination of ALK inhibitor Lorlatinib and TRAIL delayed tumour regrowth. The results herein show that cell types in bi-phasic neuroblastoma tumours have complementary drug-sensitivity profiles. Even targeting of a mutant oncoprotein inhibited only one cell type, providing an escape mechanism from targeted therapy. However, it is demonstrated that a combination treatment with an ALK inhibitor and TRAIL inhibit both lineage committed ADRN-type and precursor-like MES-type cells attenuate escape from therapy and therefore can improve clinical outcome.

Without wishing to be bound by theory, the inventors believe that the same mechanism of a mesenchymal phenotype which is responsible for a relapse applies to all ALK-positive neoplasia. This is supported by the recent finding of cells characterized by a mesenchymal phenotype is also present in non-small-cell lung cancer (Fukuda et al. Feb. 8, 2019; DOI: 10.1158/0008-5472.CAN-18-2052).

The invention therefore provides a pharmaceutical product comprising therapeutically effective amounts of an ALK inhibitor or an antibody-drug conjugate directed to the ALK receptor and a TNF-related apoptosis-inducing ligand (TRAIL) receptor agonist. The advantage thereof is that this pharmaceutical product is suitable for preventing or treating relapses in ALK positive neoplasia.

Preferably said ALK inhibitor is Lorlatinib. In another preferred embodiment, said ALK inhibitor is Alectinib.

The invention further provides the pharmaceutical product according to the invention for use in the treatment of an ALK-positive neoplasm. Preferably, said neoplasm is a malignant neoplasm. Preferably, said ALK-positive neoplasm comprises anaplastic large cell lymphoma, non-small-cell lung cancer, inflammatory myofibroblastic tumors, neuroblastoma and colorectal cancer. In a highly preferred embodiment, said an ALK-positive neoplasia is neuroblastoma or non-small-cell lung cancer. In a highly preferred embodiment, said ALK positive neoplasm is neuroblastoma. Preferably, said ALK-positive neoplasia has at least one oncogenic mutation in the ALK gene. Preferably, said ALK-positive neoplasia is characterized by the presence of tumor cells, preferably neuroblastoma cells, lacking ALK expression. Without wishing to be bound by theory, the inventors believe that such cells represent a subset of the ALK-positive neoplasia which are not responsive to a treatment targeting ALK, but are sensitive to a TRAIL receptor agonist. Preferably, said ALK-positive neoplasm is resistant to at least one ALK inhibitor or to an antibody-drug conjugate directed to the ALK receptor.

### BRIEF DESCRIPTION OF THE FIGURES

FIG. 1: MES-type neuroblastoma cells do not express ALK.

a) Box plots of mRNA expression for ALK in MES (n=8) and ADRN (n=28) neuroblastoma cell lines (**p<0.01; ANOVA). Circles indicate the values of individual cell lines. Whiskers denote the interval within 1.5 times the interquartile range (box edges) of the median (centre line). b) Neuroblastoma cell lines (n=36) plotted according to their MES- or ADRN-signature scores⁵ and their level of ALK mRNA expression (²log scale). The ALK-mutant cell line pairs are numbered. (1=SH-EP2 and 2=SH-SY5; both ALK-F1174L, 3=NBLW-MES, 4=NBLW-ADRN; both ALK-R1275L). c) The super-enhancer region associated with the ALK locus (29.28 MB-30.2 MB of chromosome 2). H3K27ac profiles for the four isogenic MES (bottom) and ADRN (top) cell line pairs. The y axis represents the number of reads per 20 million mapped reads. The ADRN-specific super-enhancer (SE) region is indicated below⁵. d) Western blot analysis of ALK, ADRN-markers (PHOX2A, PHOX2B, DBH, TH, DLK1) and MES-markers (PRRX1, FN1, HES1, VIM, SNAI2) in four isogenic cell line pairs. β-actin was used as loading control.

FIG. 2: Reprogramming of ADRN cells into MES-type cells silences ALK and induces resistance to Lorlatinib.

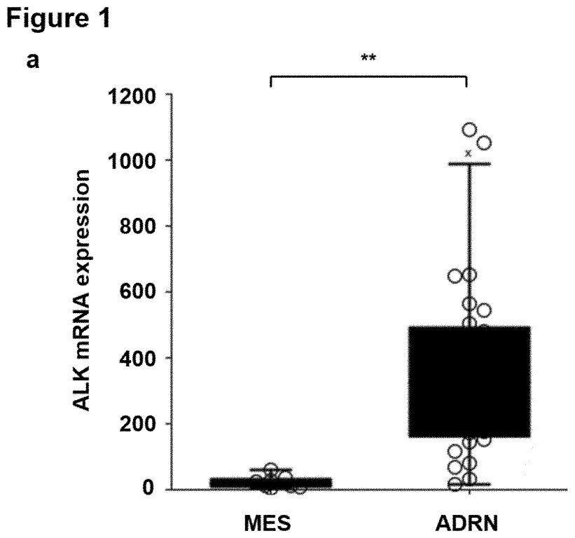

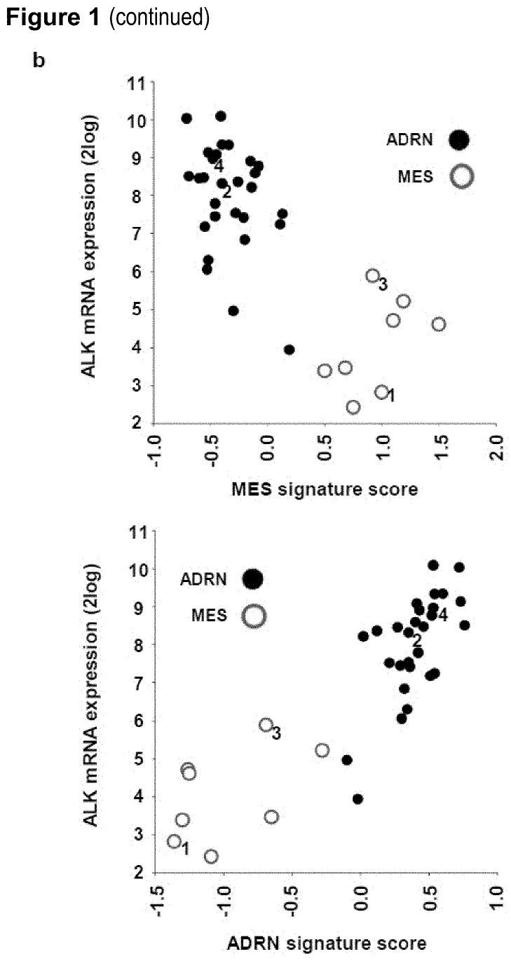

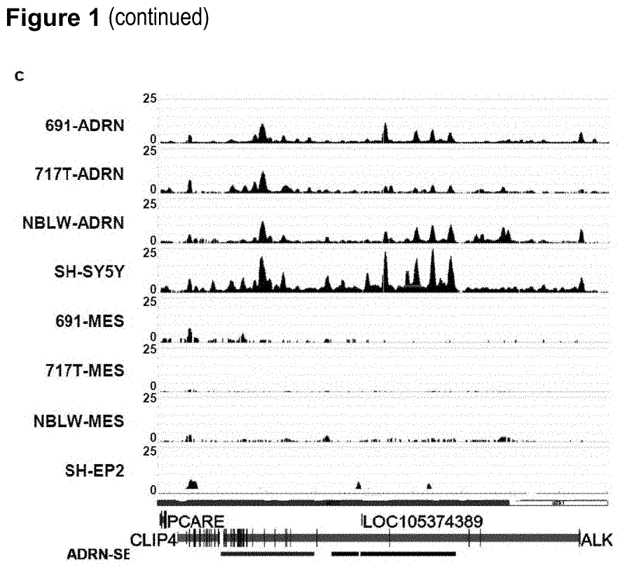

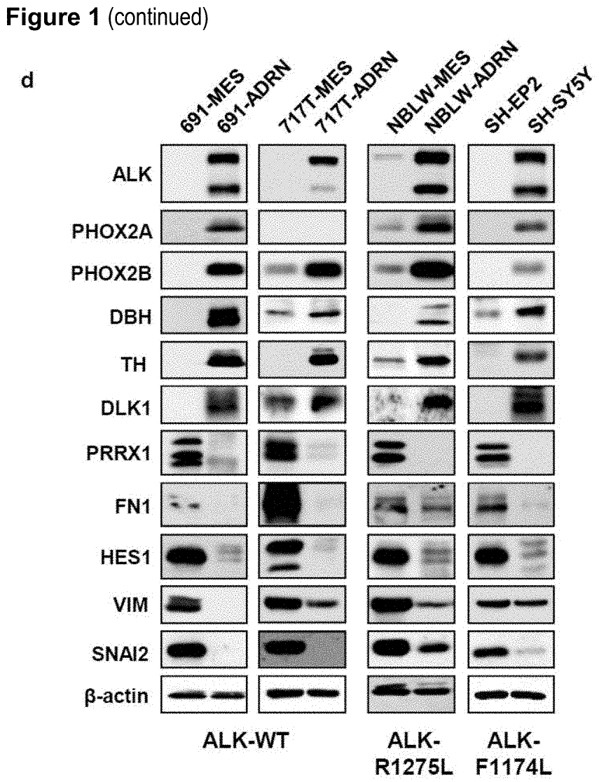

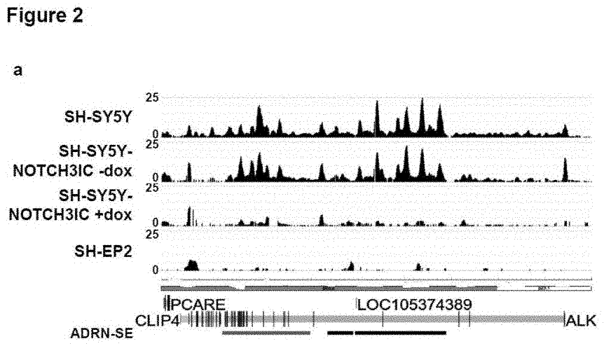

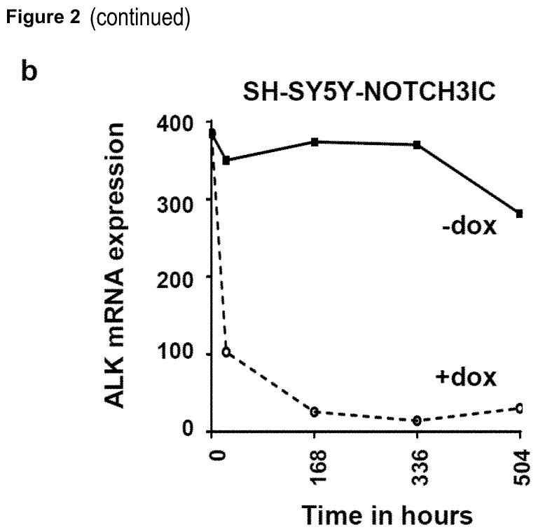

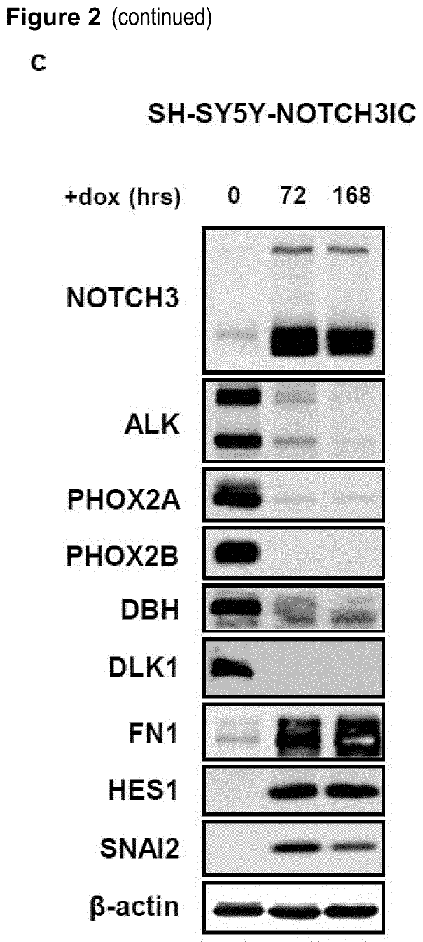

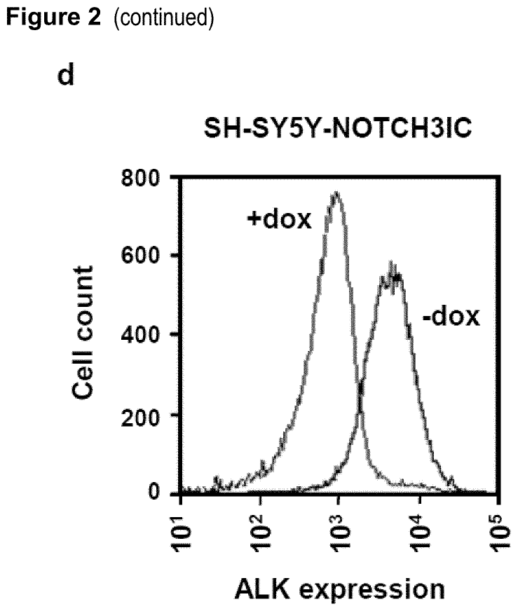

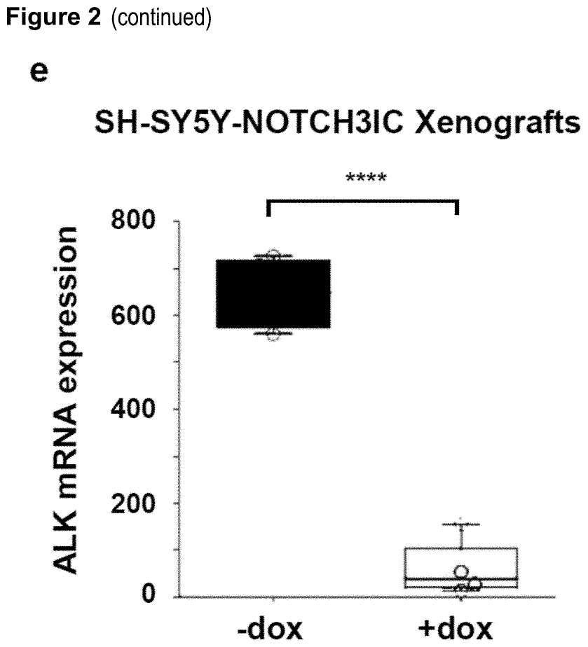

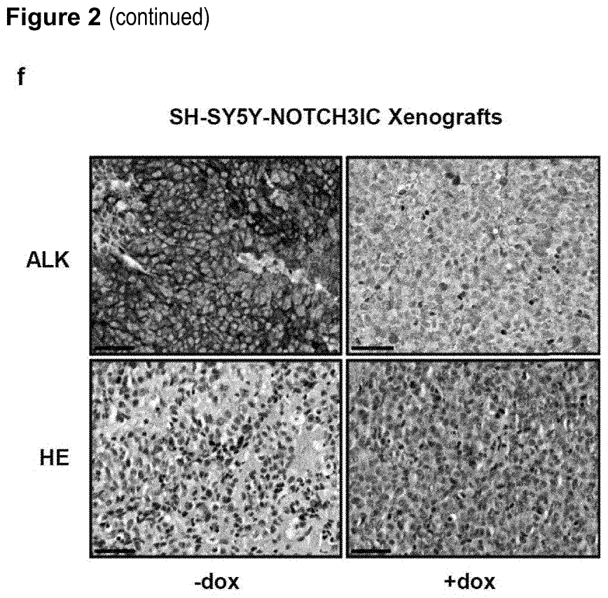

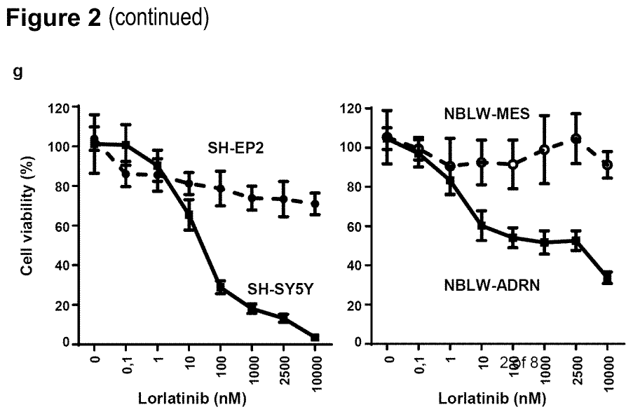

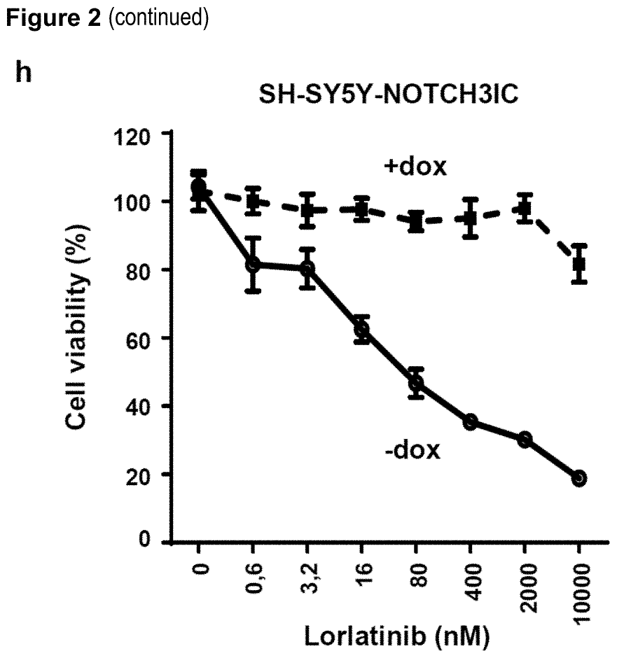

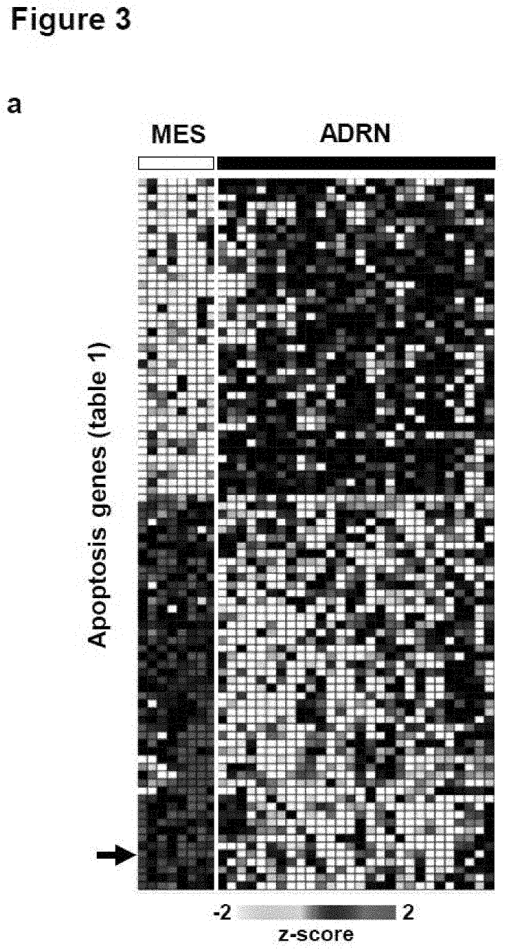

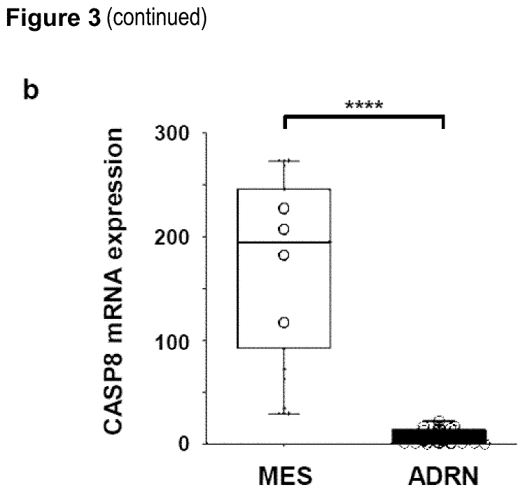

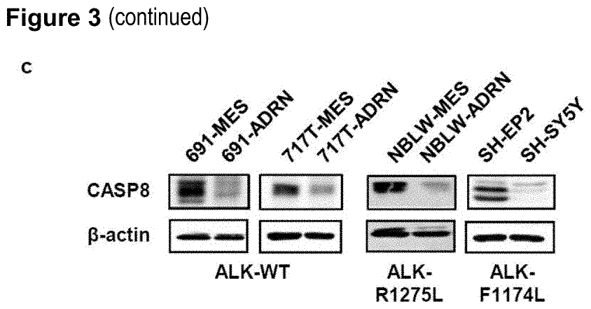

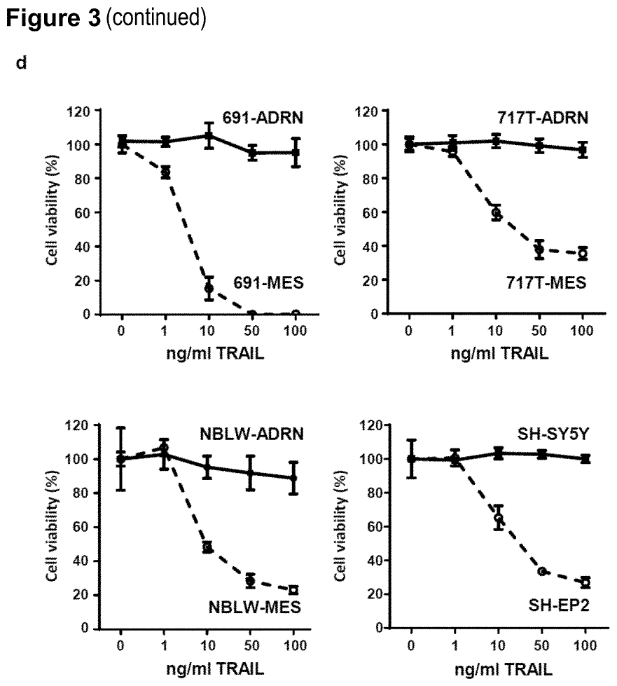

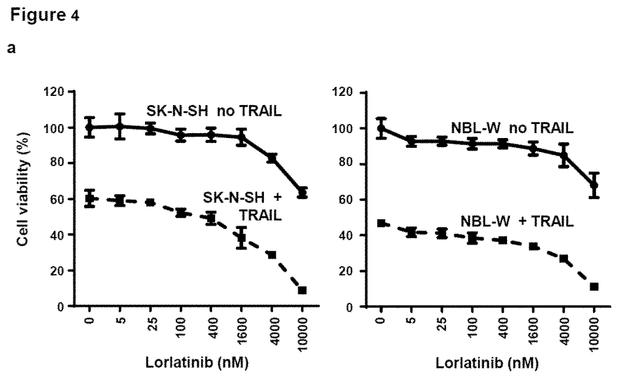

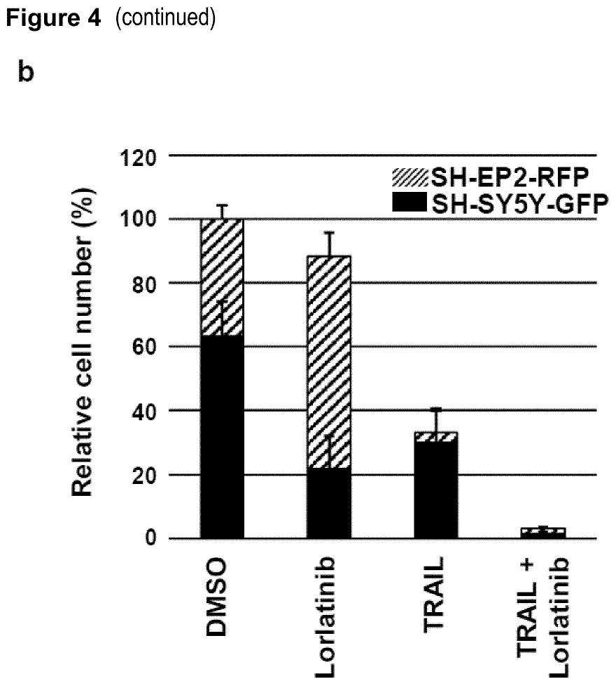

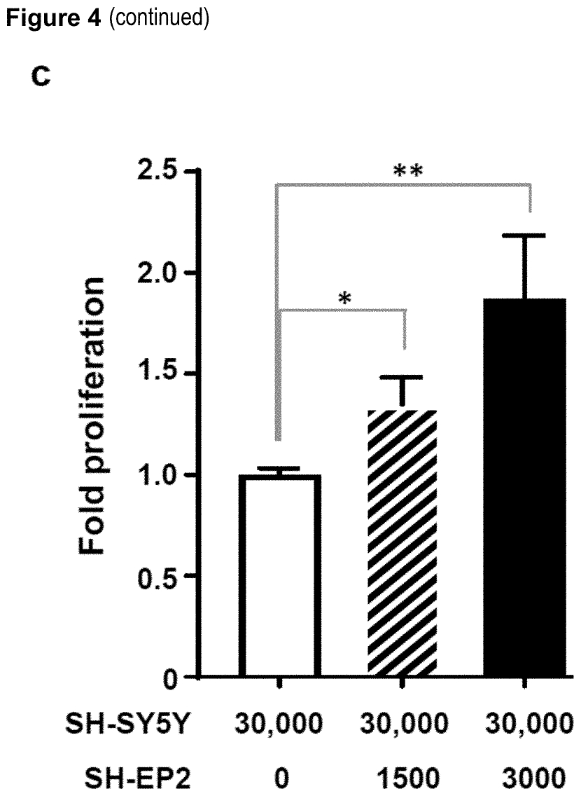

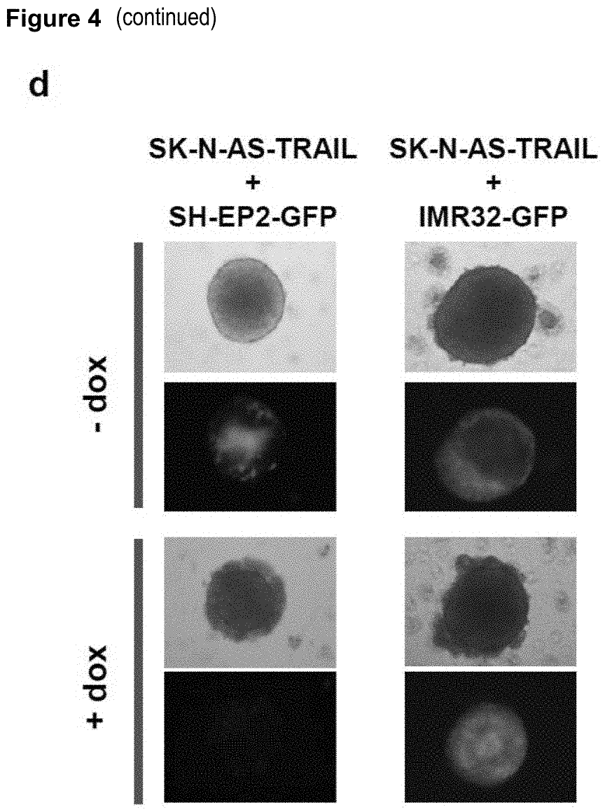

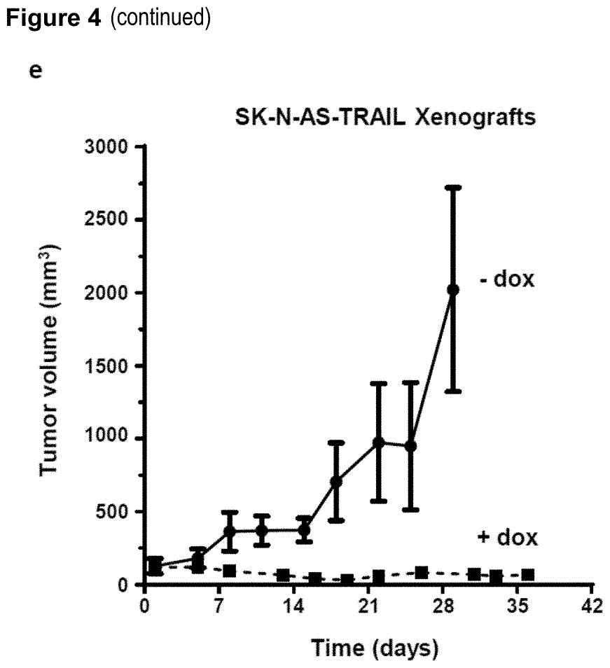

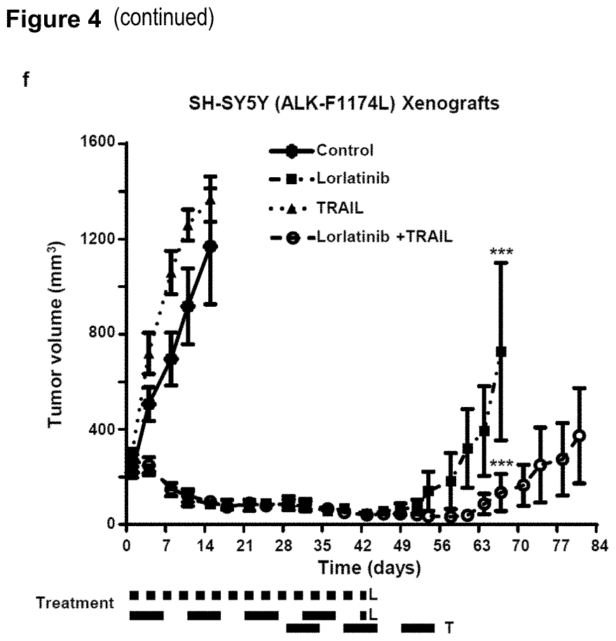

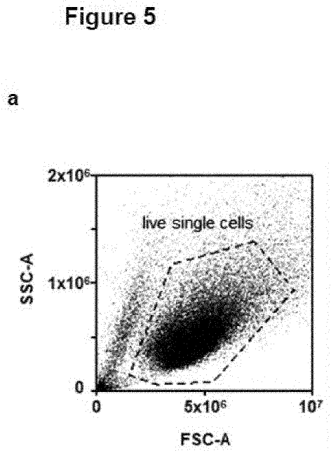

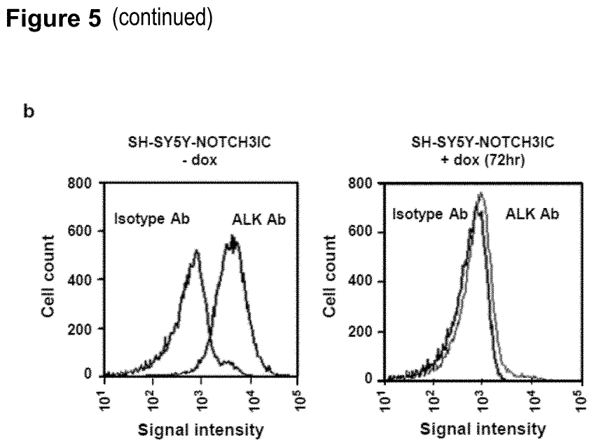

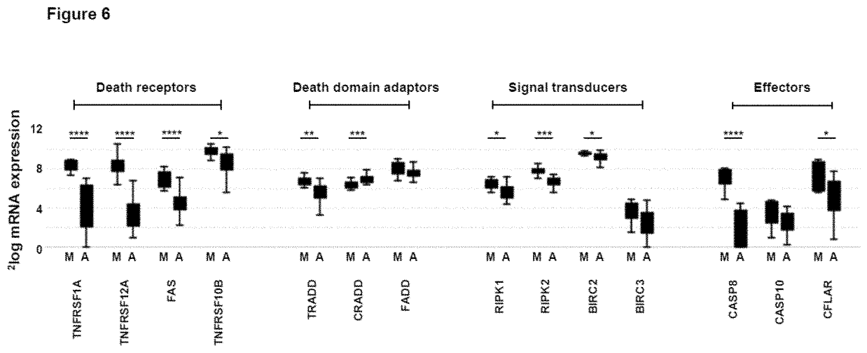

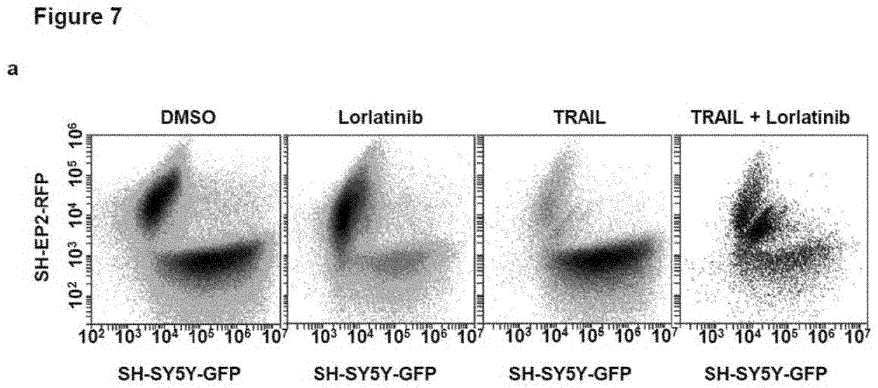

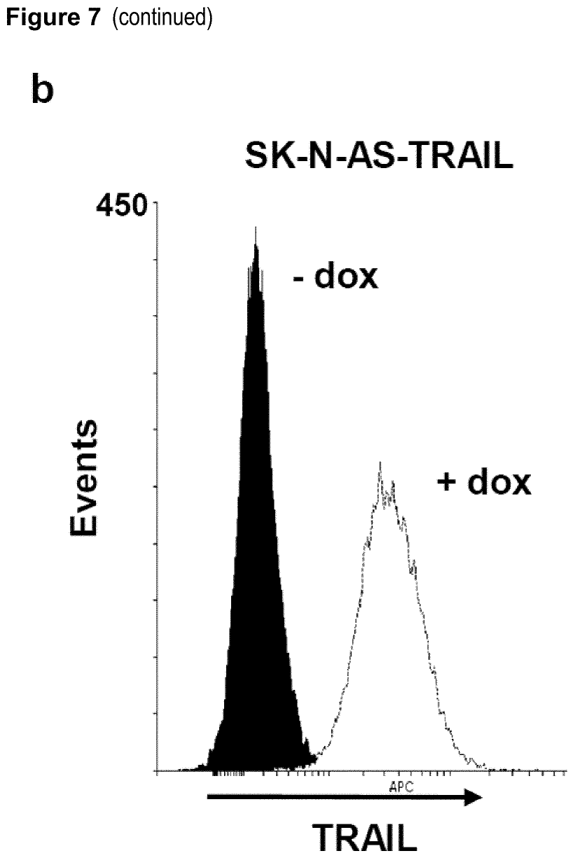

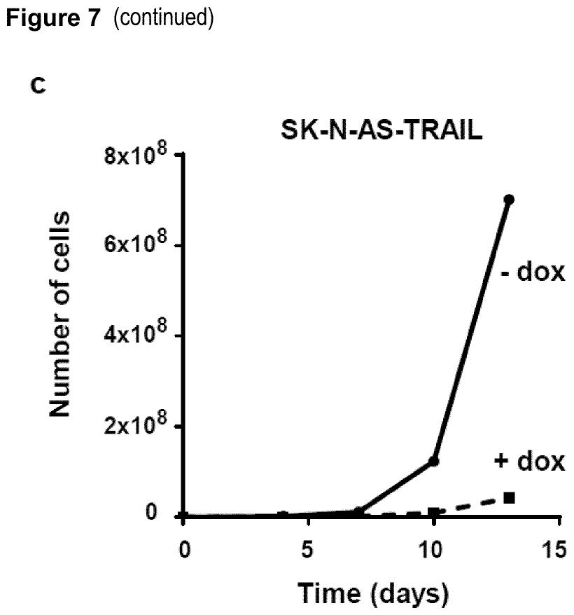

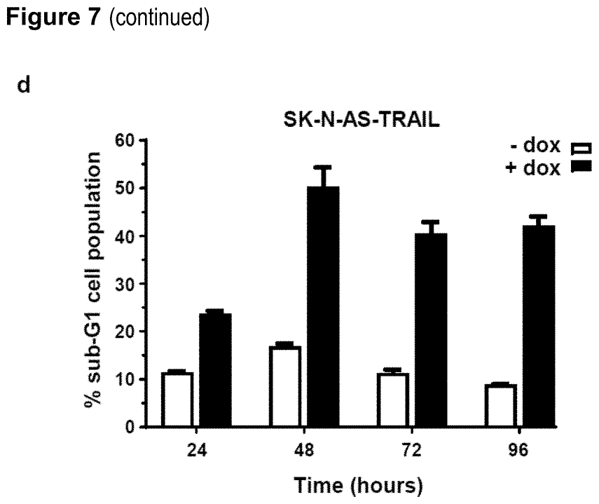

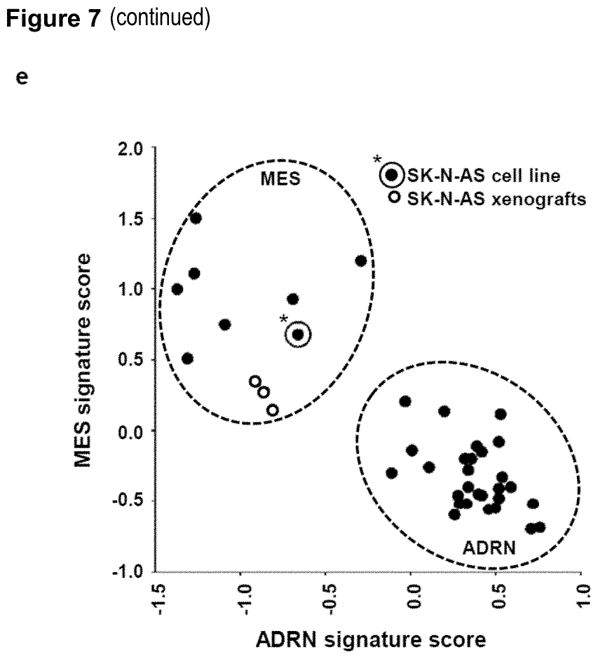

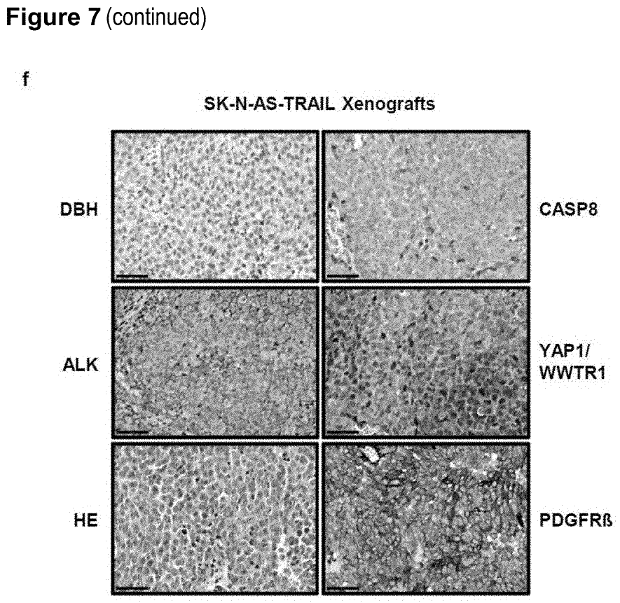

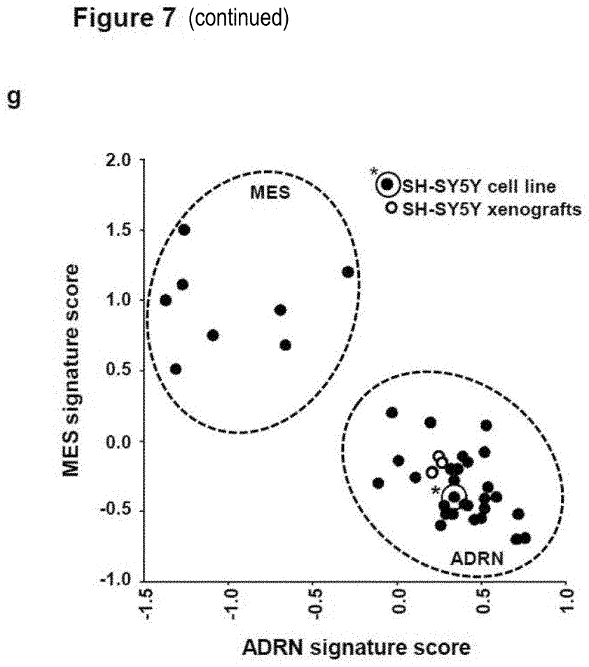

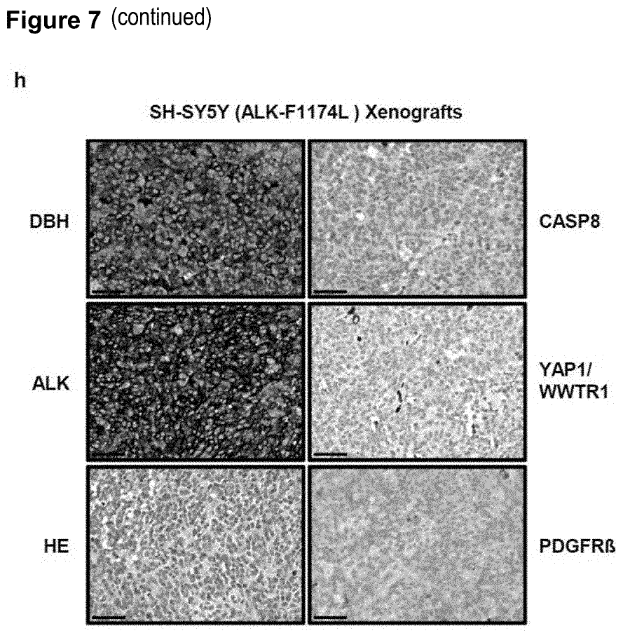

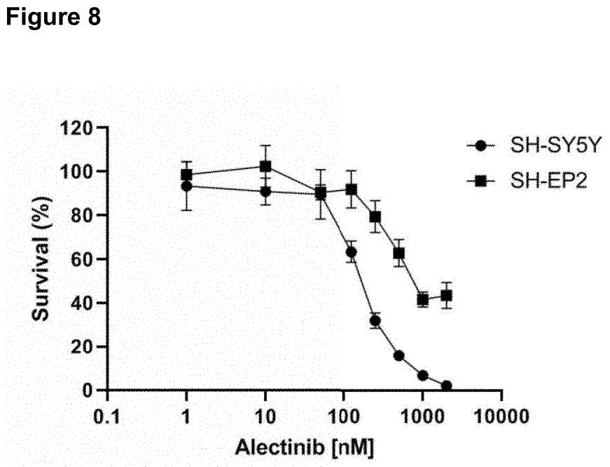

a) H3K27ac super-enhancer profiles of the ALK locus (same region as FIG. 1b) of the isogenic cell line pair SH-EP2 and SH-SY5Y and of SH-SY5Y-NOTCH3-IC cells with or without dox-induced NOTCH3-IC expression. b) Time course analysis of ALK expression in SH-SY5Y-NOTCH3-IC cells with or without dox-induced NOTCH3-IC expression. c) Western blot analysis of ALK plus ADRN- and MES-markers of SH-SY5Y-NOTCH3-IC cells after 0, 72 or 168 hours of dox-induced NOTCH3-IC expression. d) Flow cytometry analysis of ALK membrane expression (AF-647) of SH-SY5Y-NOTCH3-IC cells with or without 72 hours of dox-induced NOTCH3-IC expression. e) ALK expression in SY5Y-NOTCH3-IC xenograft tumours with NOTCH3-IC induction (n=4) as compared to non-induced control tumours (n=4) (****p<0.0001; ANOVA). f) IHC analysis of ALK expression in xenograft tumours of SH-SY5Y-NOTCH3-IC treated for 7 days with or without doxycycline. Scale bar, 50 μm. g) Sensitivity to Lorlatinib of two isogenic pairs of MES- (dotted) and ADRN-type cells (black) with mutant ALK. Cell viability was assayed after 120 hours of Lorlatinib treatment. Whiskers denote the standard error of mean (SEM; n=6). h) Sensitivity to Lorlatinib of the SH-SY5Y-NOTCH3-IC cells with or without dox-induced expression of NOTCH3-IC. Whiskers denote the SEM (n=6).

FIG. 3: MES-type neuroblastoma cells are sensitive to TRAIL.

a) Unsupervised clustering analysis of mRNA expression of the 90 genes of the apoptosis pathway (GO; 97190) that are differentially expressed between MES and ADRN cell lines (Extended data table 1). The eight MES cell lines cluster on the left (white) and the twenty-eight ADRN cell lines (black) cluster on the right (ranking cell lines and genes in Extended data table 2). z-score of expression. CASP8 is indicated by the arrow. b) Box plots of mRNA expression for CASP8 in the MES (n=8) and ADRN (n=28) neuroblastoma cell lines (****p<0.0001; ANOVA). Whiskers denote the interval as in FIG. 1a. c) Western blot analysis of CASP8 expression in the four isogenic cell line pairs. d) Sensitivity to a concentration range of TRAIL of the four isogenic pairs of MES (dotted) and ADRN cell lines (black). Viability was measured after 72 hours of TRAIL treatment. Whiskers denote the SEM (n=6).

FIG. 4: Combination treatment of TRAIL and Lorlatinib of cell lines and xenografts with a heterogeneous MES and ADRN composition.

a) Sensitivity to Lorlatinib with (dotted) or without (black) 100 ng/ml of TRAIL of the cell lines SK-N-SH (ALK-F1174L) and NBL-W (ALK-R1275L). Cell viability was measured after 120 hours. Whiskers denote the SEM (n=3). b) Flow cytometry analysis of mixed cultures of SH-EP2-RFP and SH-SY5Y-GFP cells after 120 hours of treatment. The bars indicate the surviving population of ADRN cells (black) and MES cells (striped), relative to the DMSO control (100%). Whiskers denote the SEM (n=3). c) Flow cytometry analysis of SH-SY5Y-GFP (30,000 cells/well) seeded with 0 or 1,500 or 3,000 SH-EP2-RFP cells. Y-axis depicts fold proliferation after 96 hours as compared to SH-SY5Y alone (*p<0.05, **p<0.01; unpaired t-test). Whiskers denote the SEM (n=3). d) Bright-field and fluorescent images of 3D cultures of SK-N-AS-TRAIL cells with or without doxycycline-induced TRAIL expression, co-cultured with either GFP-labelled SH-EP2 cells (MES) or GFP-labelled IMR32 cells (ADRN). e) Growth of SK-N-AS-TRAIL xenografts with (+dox, dotted) or without (−dox, black) TRAIL expression. Error bars represent the SEM (−dox; n=4 and +dox; n=5). f) Growth of xenografts of the SH-SY5Y cell line treated with vehicle (n=8), TRAIL for 4 weeks (n=8), Lorlatinib for 6 weeks (n=7) or the combination of Lorlatinib and TRAIL (n=8). Treatment schedules for Lorlatinib (L) or Lorlatinib and TRAIL (T) are indicated below the figure. Error bars represent SEM (***p<0.001; ANOVA).

FIG. 5 shows that Reprogramming to MES-type induces downregulation of ALK. Flow cytometry analysis of ALK protein on the cell membrane on SH-SY5Y-NOTCH3-IC cells without doxycycline treatment (−dox, left panel) as compared to an isotype control. Similar in the right panel on SH-SY5Y-NOTCH3-IC cells with (+dox) 72 hours doxycycline-induced expression of NOTCH3-IC. Signal intensity measure at 647 nm is depicted on the x-axis; cell counts are depicted on the y-axis.

FIG. 6: Genes of the extrinsic apoptosis pathway are preferentially expressed in MES-type neuroblastoma cells. Expression of extrinsic apoptosis pathway genes in MES (M, n=8) and ADRN (A, n=28) cell lines (*p<0.05; **p<0.01; ***p<0.001; ****p<0.0001; ANOVA). Box plots show ²log transformed mRNA expression values as determined by Affymetrix profiling. Whiskers denote the interval within 1.5 times the interquartile range (box limits) of the median (center line).

FIG. 7: Targeting MES-type and ADRN-type neuroblastoma cells with TRAIL and Lorlatinib

a) Flow cytometry analysis of remaining cell populations of mixed cultures of SH-EP2-RFP and SH-SY5Y-GFP cells treated with either DMSO, Lorlatinib (5 μM), TRAIL (100 ng/ml) or both for 120 hours (representative of triplicate). X-axis: GFP signal intensity; Y-axis: RFP signal intensity. b) Flow cytometry analysis of TRAIL on the cell membrane on SK-N-AS-TRAIL cells with (dark) or without (light) 24 hours doxycycline-induced expression of TRAIL. X-axis: Signal intensity; Y-axis: cell counts. c) 3T3 assay performed on the SK-N-AS-TRAIL cells with (dark) or without (light) doxycycline-induced expression of TRAIL (n=3). X-axis: Signal intensity; Y-axis: cell counts. Error bars represent standard deviation. d) Flow cytometry analysis of propidium-iodide stained SK-N-AS-TRAIL cells with (dark) or without (light) doxycycline-induced TRAIL expression. The y-axis depicts the percentage of cells in the sub-G1 fraction after 24, 48, 72 or 96 hours. e) Neuroblastoma cell lines (n=36) plotted according to their MES- and ADRN-signature scores as shown in FIG. 1b. The SK-N-AS cell line is marked with an asterix. Super-imposed are three SK-N-AS-TRAIL xenografts analysed by Affymetrix mRNA profiling and positioned according to their MES and ADRN signature scores. f) Immunohistochemistry of a xenograft of SK-N-AS-TRAIL cells with ADRN-markers (ALK and DBH) and MES-markers (CASP8, YAP1/WWTR1 and PDGFRβ). Scale bar, 50 μM. g) Neuroblastoma cell lines (n=36) plotted according to their MES- and ADRN-signature scores as shown in FIG. 1b. The SH-SY5Y cell line is marked with an asterix. Super-imposed are three SH-SY5Y xenografts analysed by Affymetrix mRNA profiling and positioned according to their MES and ADRN signature scores. h) Immunohistochemistry of a xenograft of SH-SY5Y cells for ADRN-markers (ALK and DBH) and MES-markers (CASP8, YAP1/WWTR1 and PDGFRβ). Scale bar, 50 μM.

FIG. 8: Reprogramming of ADRN cells into MES-type cells silences ALK and induces resistance to Alectinib.

MES-type neuroblastoma cells are more resistant to the inhibitor of mutated ALK protein Alectinib (Roche) than ADRN-type neuroblastoma cells. Sensitivity to Alectinib was measured in an isogenic pair of MES-type (black squares) and ADRN-type (black dots) cells with mutant ALK. Cell survival was assayed after 120 hours of Alectinib treatment. Whiskers denote the standard error of mean (SEM; n=6).

### DETAILED DESCRIPTION

### Definitions

As used herein, the term “about” means a range around a given value wherein the resulting value is substantially the same as the expressly recited value. In one embodiment, “about” means within 25% of a given value or range. For example, the phrase “about 70% by weight” comprises at least all values from 52% to 88% by weight. In another embodiment, the term “about” means within 10% of a given value or range. For example, the phrase “about 70% by weight” comprises at least all values from 63% to 77% by weight. In another embodiment, the term “about” means within 7% of a given value or range. For example, the phrase “about 70% by weight” comprises at least all values from 65% to 75% by weight.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range was explicitly recited.

As used herein, the terms “therapies” and “therapy” can refer to any protocol(s), method(s), compositions, formulations, and/or agent(s) that can be used in the prevention, treatment, management, or amelioration of a condition or disorder or one or more symptoms thereof (e.g., one or more symptoms or one or more conditions associated therewith).

In certain embodiments, the terms “therapies” and “therapy” refer to drug therapy such as chemotherapy, adjuvant therapy, radiation, surgery, biological therapy, supportive therapy and/or other therapies useful in treatment, management, prevention, or amelioration of a condition or disorder or one or more symptoms thereof (e.g., one or more symptoms or one or more conditions associated therewith). In certain embodiments, the term “therapy” refers to a therapy other than the combination product or kit described herein. In specific embodiments, an “additional therapy” and “additional therapies” refer to a therapy other than a treatment using the combination product or kit described herein. In a specific embodiment, a therapy includes the use of the combination product or kit described herein as an adjuvant therapy. For example, using the combination product or kit described herein in conjunction with a drug therapy such as chemotherapy, biological therapy, surgery, supportive therapy and/or other therapies useful in treatment, management, prevention, or amelioration of a condition or disorder or one or more symptoms thereof (e.g., one or more symptoms or one or more conditions associated therewith).

The present invention is based on the finding that MES cells are resistant to the ALK inhibitor Lorlatinib, whereas the ADRN-type cells were highly sensitive to Lorlatinib. Surprisingly, when tested with a combined treatment comprising TRAIL and Lorlatinib in two neuroblastoma cell lines with heterogeneous MES and ADRN composition: SK-N-SH and NBL-W, each of the parental cell lines was partly killed by a Lorlatinib-only or a TRAIL-only treatment, but a combination treatment killed almost 100% of the cells (see FIG. 4a). To quantify treatment effects, the inventors reconstituted a mixed cell line by co-culturing of GFP-labelled SH-SY5Y cells (ADRN-type) and RFP-labelled SH-EP2 cells (MES-type). FACS analysis quantified how TRAIL strongly reduced the MES population and Lorlatinib reduced the ADRN population (FIG. 4b and FIG. 7a). Combination of Lorlatinib and TRAIL eliminated 97% of the cells (FIG. 4b). Remarkably, inhibition of MES cells by TRAIL did not only affect the MES population, but also resulted in a reduction of the ADRN population. The inventors therefore investigated whether the proliferation rate of ADRN-type SH-SY5Y cells is stimulated by the MES-type SH-EP2 cells. Addition of 3000 SH-EP2 cells to a population of 30,000 SH-SY5Y cells gave 1.8-fold increase of the SH-SY5Y population as compared to the control, confirming that these MES cells stimulate their ADRN-type counterparts (FIG. 4c). Combination therapy with TRAIL and Lorlatinib is therefore effective to heterogeneous neuroblastoma populations in vitro.

The inventors subsequently tested the effect of TRAIL in a mouse xenograft model. The inventors first tested TRAIL in the neuroblastoma cell line SK-N-AS which has a MES phenotype and forms predominantly MES-type tumours after xenografting. The inventors generated SK-N-AS cells with a Doxycycline inducible recombinant TRAIL transgene. Doxycycline treatment induced strong TRAIL expression on their cell membranes and results in apoptosis of a large proportion of the cells in vitro (FIG. 7b-d). The inventors tested whether this proceeds via activation of membrane-expressed TRAIL receptors. The inventors co-cultured the SK-N-AS-TRAIL cells with either GFP-labelled IMR32 or SH-EP2 neuroblastoma cells in a 3D model. Doxycycline-induced TRAIL expression killed the MES-type SH-EP2 cells but not the ADRN-type IMR32 cells, confirming the specific killing of MES cells via juxtacrine signaling (FIG. 4d). Xenografting of the SK-N-AS-TRAIL cells in mice resulted in rapidly growing tumours which maintained their MES phenotype as shown by immunohistochemical (IHC) and mRNA profiling analysis (FIGS. 7e and 7f). Induction of TRAIL by doxycycline treatment of the established tumours (200 mm³) induced a complete regression, which was maintained during the five-week doxycycline stimulation (FIG. 4e). TRAIL is therefore effective to MES-type tumour cells in vivo.

Based on the above, the present invention provides a pharmaceutical product comprising therapeutically effective amounts of an ALK inhibitor or an antibody-drug conjugate directed to the ALK receptor and a TNF-related apoptosis-inducing ligand (TRAIL) suitable for use in a combination therapy. Such pharmaceutical product is suitable for treatment of ALK-positive neoplasia.

The term “effective amount” as used herein means a dosage which is sufficient in order for the treatment of the patient to be effective compared with no treatment. Preferably an effective amount will relieve to some extent one or more of the symptoms of the disorder being treated. In reference to the treatment of the ALK positive neoplasm, preferably neuroblastoma, a therapeutically effective amount refers to that amount which has the effect of (1) reducing the size of a neuroblastoma tumor, (2) inhibiting (that is, slowing to some extent, preferably stopping) the metastasis of the ALK positive neoplasm, preferably the neuroblastoma tumor metastasis, (3) inhibiting to some extent (that is, slowing to some extent, preferably stopping) growth of the ALK positive neoplasm, preferably the neuroblastoma tumor growth, (4) preventing or delaying relapses and/or, (5) relieving to some extent (or, preferably, eliminating) one or more symptoms associated with the ALK positive neoplasm, preferably neuroblastoma.

The term “combination” applied to active ingredients is used herein to define a single pharmaceutical composition (formulation) comprising both drugs of the invention (i.e., an ALK inhibitor or an antibody-drug conjugate directed to the ALK receptor and a TNF-related apoptosis-inducing ligand receptor agonist) or two separate pharmaceutical compositions (formulations), each comprising a single drug of the invention (i.e., an an ALK inhibitor or a TNF-related apoptosis-inducing ligand receptor agonist), to be administered conjointly.

Pharmaceutical compositions of the present invention may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

Within the meaning of the present invention, the term “conjoint administration” is used to refer to administration of (a) the ALK inhibitor or the antibody-drug conjugate directed to the ALK receptor and (b) TRAIL receptor agonist simultaneously in one composition, or simultaneously in different compositions, or sequentially. For the sequential administration to be considered “conjoint”, however, the (a) ALK inhibitor or the antibody-drug conjugate directed to the ALK receptor, and (b) TRAIL receptor agonist must be administered separated by a time interval that still permits the resultant beneficial effect for treating, preventing, arresting, delaying the onset of and/or reducing the risk of developing ALK-positive neoplasm in a mammal. For example, the (a) ALK inhibitor antibody-drug conjugate directed to the ALK receptor and (b) TRAIL receptor agonist can be administered as disclosed herein in a scheme of 4 weeks of ALK inhibitor, followed by 2 weeks of a combination of the (a) ALK inhibitor antibody-drug conjugate directed to the ALK receptor and (b) TRAIL receptor agonist and subsequently 2 weeks of TRAIL receptor agonist. As used herein, the term “neuroblastoma” means a tumor of the postganglionic sympathetic nervous system.

The term “ALK inhibiting agent” or “ALK inhibitor,” as used herein, refers to any compound that can inhibit the biological activity of an ALK tyrosine kinase. In some embodiments the said ALK inhibitor is a proteolysis-targeting chimaera (PROTAC), as disclosed in Scudellari M., Nature. 2019 March; 567(7748):298-300. doi: 10.1038/d41586-019-00879-3. Suitable ALK inhibitors include inhibitors of ALK fusion proteins and/or ALK point mutation variants. Inhibition of ALK leads to the disruption of ALK-mediated signaling and the inhibition of cell growth in ALK-expressing tumor cells. Exemplary ALK inhibiting agents include, but are not limited to, PF-02341066, PDD, 2-methyl-11-(2-methylpropyl)-4-oxo-4,5,6,11,12,13-hexahydro-2H-indazolo[5,4-a]pyrrolo[3,4-c]carbazol-8-yl[4-(dimethylamino)benzyl]carbamate, (1S,2S,3R,4R)-3-({5-chloro-2-[(1-ethyl-2,3,4,5-tetrahydro-6-methoxy-2-oxo-1H-1-benzazepin-7-yl)amino]-4-pyrimidinyl}amino)bicyclo[2.2.1]hept-5-ene-2-carboxamide, and NVP-TAE684 (CAS 761439-42-3) see, for example, PNAS 104:270-275, 2007; Choi, Y. L. et al. (2008) Cancer Res. 68:4971-2976; and Biochemistry 48:3600-3609, 2009, which are hereby incorporated by reference). In a preferred embodiment, said ALK inhibitor comprises crizotinib (also a ROS1 inhibitor), ceritinib, alectinib, brigatinib (also an EGFR inhibitor) or lorlatinib. In a preferred embodiment said ALK inhibitor is lorlatinib or alectinib.

As used herein, the term “antibody-drug conjugate directed to the ALK receptor” as used herein refers to any conjugate comprising an antibody which binds to the ALK receptor and a toxin which is capable of killing the cell which expresses the ALK receptor to which it is bound. Suitable antibody-drug conjugates directed to the ALK receptor are disclosed in Sano et al. Science Translational Medicine 13 Mar. 2019 Vol. 11, Issue 483.

### TRAIL Receptor Agonists

A TRAIL receptor agonist is an agent that binds to a TRAIL receptor, such as TRAIL receptor 1 (TRAIL R1, also known as “death receptor 4” or DR4), TRAIL receptor 2 (TRAIL R2, also known as “death receptor 5” or DR5), or both DR4 and D5, and leads to apoptosis in at least one mammalian (e.g., human) cell type (such as a TRAIL-sensitive tumor cell line) when used in an amount effective to induce apoptosis under physiological conditions. TRAIL receptor agonists according to the invention encompass antibodies which require cross-linking for in vitro apoptotic activity.

Binding to a TRAIL receptor can be assessed by flow cytometry. Functional activity of a TRAIL receptor agonist can be monitored by measurement of apoptosis induction in Colo205 cells using standard cell viability and caspase activation assays. In some embodiments, TRAIL receptor agonists bind to DR4 and/or DR5 as assessed by flow cytometry and result in a 60-100% decrease in cell viability and a 5-12 fold increase in caspase 3 activity. In some embodiments the TRAIL receptor agonist is a soluble TRAIL polypeptide.

### Flow Cytometry

Binding of an agent to a TRAIL receptor is detected by flow cytometry. In one embodiment, Colo205 cells are incubated in blocking buffer (10% FBS/5% goat serum/FACS buffer), washed in FACS buffer (1% FBS/PBS), and resuspended in 10 μg/ml primary antibody (DR4 or TRAIL-R1 and/or DR5 or TRAIL-R2 antibodies, which are commercially available, e.g. from eBiosciences, San Diego, Calif.) or isotype control for 45 min at 4° C. After washing, cells are resuspended in 10 μg/ml biotinylated secondary antibody for 45 min at 4° C., washed again and incubated with 10 μg/ml streptavidin-phycoerythrin (BD Pharmingen, San Diego, Calif.) for 45 min at 4° C. Cells are then analyzed on a flow cytometer. TRAIL receptor agonist binding may also be shown by measuring binding affinity of an agonist to the extracellular domain of DR4 (TRAIL-R1) and/or DR5 (TRAIL-R2) protein immobilized on a 96-well plate using an ELISA based format, as described in (Pukac et al., Br. J. Cancer 92, 1430-41, 2005).

### Measurement of Apoptosis Induction

To monitor cell viability, Colo205 cells are plated at 2×104 cells/well and treated with a TRAIL receptor agonist at a range of concentrations relevant for the modality of the agonist (antibody, recombinant protein) as described in Gliniak, Cancer Res. 59, 6153-58, 1999; Motoki et al., Clin. Cancer Res. 11, 3126-35, 2005; or Pukac et al., 2005. After 24-48 hours, viability is assessed by detecting the percentage of dead cells in a well.

### Caspase 3 Activity Assay

For a caspase 3 activity assay, Colo205 cells are plated at 1.8×104 cells/well and treated with a TRAIL receptor agonist at a range of concentrations relevant for the modality of the agonist (antibody, recombinant protein) as described in Gliniak, Cancer Res. 59, 6153-58, 1999; Motoki et al., Clin. Cancer Res. 11, 3126-35, 2005; or Pukac et al., 2005. After 5-18 hr, caspase 3 activity is measured (for example, using the Caspase Glo 3/7 assay (Promega, Madison, Wis.) according to manufacturer's protocol).

Suitable TRAIL receptor agonists include TRAIL itself (including recombinant TRAIL), as well as antibodies (particularly agonist monoclonal antibodies), peptibodies, avimers, peptide-mimetic compounds, small molecules, and proteins. The monoclonal antibodies HGS-ETR1 (“mapatumumab,” DR4 antibody), HGS-ETR2 (“lexatumumab,” DR5 antibody), HGS-TR2J (DR5 antibody), Apomab (human DR5 antibody), recombinant human Apo2L/TRAIL (which activates both DR4 and DR5), CS-1008 (humanized DR5 antibody), AMG 655 (DR5 antibody), LBY135 (DR5 antibody), and TR8 (DR5 antibody) are examples of TRAIL receptor agonists. See also U.S. Pat. Nos. 7,115,717; 7,361,341; US 2007/0292411;US 2005/0249729; and US 2002/0155109.

In some preferred embodiments, said TRAIL receptor agonist is a recombinant human Apo2L/TRAIL and monoclonal agonist antibody directed against death receptors-4 (DR4) or -5 (DR5). In another preferred embodiment, said TRAIL receptor agonist is a human TRAIL or a recombinant TRAIL, preferably human, or multimeres of derivatives of TRAIL proteins, including APG350 as disclosed in Legler et al., Cell Death Dis. 2018 May 1; 9(5):445) and ABBV-621. Agonistic antibodies are disclosed in GONG J, YANG D, KOHANIM S, et al. Mol Cancer Ther. 2006; 5(12):2991-3000 and in ZENG Y, WU XX, FISCELLA M, et al. Int J Oncol. 2006; 28(2):421-430).

In another embodiment said TRAIL receptor agonist is a TRAIL short (TRAIL-s) as disclosed in U.S. Pat. No. 8,008,261B2, which is a polypeptide comprising, or consists essentially of, an amino acid sequence having 80% identity to the sequence set forth in SEQ ID NO:1 or 2 as disclosed in U.S. Pat. No. 8,008,261B2.

In another preferred embodiment, said TRAIL receptor agonist is a TRAIL polypeptide as defined in WO2012117336 (A2), page 18 line 13 and further.

In another preferred embodiment, said TRAIL receptor agonist is a TRAIL DR agonist as defined in any of the claims of WO2012117336 (A2).

Most human neuroblastoma tumours include at diagnosis only a low percentage of MES-type cells, which were proposed to preferentially survive therapy and may play a role in relapse development⁵. The inventors tested whether eradication of MES cells in a predominantly ADRN-type neuroblastoma would delay relapse development. SH-SY5Y cells have an ADRN phenotype in vitro and form rapidly growing xenografts mainly consisting of ADRN cells (FIGS. 7g and 7h). A 4-week treatment with TRAIL alone did not inhibit tumour growth and even induced a slight acceleration (FIG. 4f). A six-week treatment with Lorlatinib alone induced rapid regression of the tumours to marginally palpable rests. After cessation of Lorlatinib treatment, about half of the tumours relapsed after 7 to 16 days, which confirms a previous report¹². Combined treatment with Lorlatinib and TRAIL also induced rapid tumour regression, only followed by relapses in about half of the mice. Further, the addition of TRAIL to the Lorlatinib treatment delayed tumour outgrowth with about two weeks, as evident by a significant (p<0.001; ANOVA) reduction of the average tumour volume at day 67 (FIG. 4f). These data show that targeting of the minor population of MES cells in neuroblastoma can abate tumour regrowth in animals.

The invention therefore further provides the pharmaceutical product according to the invention for use in the treatment of an ALK positive neoplasm in a mammal. Preferably said mammal is a human.

As used herein, the term “ALK-positive neoplasm” refers to a malignancy which is characterized by the expression of the ALK gene in at least part of its cells. ALK has been recognized as a therapeutic target in ALK-positive neoplasia including anaplastic large cell lymphoma, non-small-cell lung cancer, inflammatory myofibroblastic tumors, neuroblastoma and colorectal cancer. Both chromosomal rearrangements, leading to the expression of fusion kinases, and kinase-activating point mutations, have been found to trigger the oncogenic activation of ALK.

In a preferred embodiment, said ALK positive neoplasm is chemoresistant. As used herein, the term “chemoresistant” refers to a tumor or cancer cell that shows little or no significant detectable therapeutic response to an agent used in chemotherapy

In another preferred embodiment said ALK positive neoplasm is relapsing or refractory.

In another preferred embodiment, said ALK positive neoplasm is resistant to a targeted therapy, preferably a targeted therapy with an ALK inhibitor, most preferably lorlatinib or alectinib. Preferably, said ALK positive neoplasm is resistant to at least one ALK inhibitor.

In a preferred embodiment, said neuroblastoma is characterized by an oncogenic mutation in the ALK gene. Preferably, said neuroblastoma is characterized by the presence of ALK mutations in the DNA of all or part of the tumor cells and the presence in the tumor of a fraction of tumor cells lacking ALK expression. “Anaplastic lymphoma kinase” and “ALK” are used interchangeably herein and refer to native anaplastic lymphoma kinase, and certain variants and mutations thereof, derived from any source (e.g., rodents, humans, and other mammals). In some embodiments, ALK protein is represented by NCBI Ref Seq identification number NP-004295. Unless indicated otherwise, the terms refer to the human protein. The gene encoding ALK may also be referred to herein as “ALK”. In some embodiments, ALK nucleotide sequences are represented by NCBI Ref Seq identification number NM-004304.3 and GenBank accession number 29029631, relevant sequences therein (e.g., the coding, 5′ UTR, 3′UTR, transcription start, translation start, transcription stop, translation stop, etc. sequences) of which can readily be identified by a skilled artisan.

In another preferred embodiment, said ALK positive neoplasm, more preferably said neuroblastoma, is characterized by the presence of MES cells. As used herein, the term “MES cell” refers to precursor-like mesenchymal tumour cells. MES cells express CASP8 (such as NP_001073593). In a preferred embodiment, said MES cells express CASP8, YAP1/WWTR1 and/or PDGFRβ.

In a preferred embodiment, said treatment is preceded by a step wherein the ALK expression is determined in a biological sample of said patient. In another preferred embodiment, said treatment is preceded by a step wherein the CASP8, YAP1/WWTR1 and/or PDGFRβ expression is determined in a biological sample of said patient. Preferably said biological sample comprises a sample of the ALK positive neoplasm, such as a biopsy. Preferably said biological sample is a neuroblastoma sample.

### EXAMPLES

The examples and preparations provided below further illustrate and exemplify particular aspects and embodiments of the invention. It is to be understood that the scope of the present invention is not limited by the scope of the following examples.

Several clinical studies with ALK inhibitors in neuroblastoma are ongoing, including studies with the third generation ALK inhibitor Lorlatinib (PF-06463922)¹⁴. The inventors therefore tested whether MES cells are resistant to targeted ALK inhibition. The two isogenic cell line pairs with ALK mutations were treated with Lorlatinib. In both pairs, the ADRN-type cells were highly sensitive to Lorlatinib, but the MES-type counterparts were resistant and continued to proliferate (FIG. 2g). The inventors also tested the Lorlatinib response of reprogrammed MES cells. SY5Y-NOTCH3-IC cells without doxycycline treatment were sensitive to Lorlatinib, but reprogramming to the MES phenotype rendered them resistant (FIG. 2h). As MES cells were detected in most primary neuroblastoma⁵, the inventors conclude that treatment-naïve neuroblastoma may include a population that is resistant to Lorlatinib.

The resistance of MES-type cells to both chemotherapy and ALK inhibitors urged us to search for MES-specific drugs. The inventors investigated differential expression of cancer-related pathways in mRNA profiles of 36 MES and ADRN cell lines. In an apoptosis-related gene set, 90 genes showed differential expression between both cell types (FIG. 3a and table 1 and 2). Caspase 8 (CASP8), a key mediator of the extrinsic apoptosis pathway, was highly expressed in MES cells but silent in ADRN cells (FIG. 3b). Western blot analysis of the isogenic cell line pairs confirmed the exclusive expression of CASP8 in MES cells (FIG. 3c). Also other genes of the extrinsic pathway were preferentially expressed in MES cells as compared to ADRN cells (FIG. 6). The inventors tested several inducers of the extrinsic apoptosis route in the isogenic cell line pairs and found that recombinant soluble human TRAIL efficiently killed MES cells, while leaving ADRN cells unaffected (FIG. 3d). The extrinsic apoptotic route can therefore be activated in MES cells providing a therapeutic perspective to eliminate this cell population.

The inventors tested combined treatment with TRAIL and Lorlatinib in two neuroblastoma cell lines with heterogeneous MES and ADRN composition: SK-N-SH and NBL-W are the parental cell lines from which the isogenic cell line pairs SH-EP2/-SY5Y and NBLW-MES/-ADRN were sub-cloned¹⁵ and see Material and Methods). Each of the parental cell lines was partly killed by Lorlatinib-only or TRAIL-only treatment, but combination treatment killed almost 100% of the cells (FIG. 4a). To quantify treatment effects, the inventors reconstituted a mixed cell line by co-culturing of GFP-labelled SH-SY5Y cells (ADRN-type) and RFP-labelled SH-EP2 cells (MES-type). FACS analysis quantified how TRAIL strongly reduced the MES population and Lorlatinib reduced the ADRN population (FIG. 4b and FIG. 7a). Combination of Lorlatinib and TRAIL eliminated 97% of the cells (FIG. 4b). Remarkably, inhibition of MES cells by TRAIL did not only affect the MES population, but also resulted in a reduction of the ADRN population. The inventors therefore investigated whether the proliferation rate of ADRN-type SH-SY5Y cells is stimulated by the MES-type SH-EP2 cells. Addition of 3000 SH-EP2 cells to a population of 30,000 SH-SY5Y cells gave 1.8 fold increase of the SH-SY5Y population as compared to the control, confirming that these MES cells stimulate their ADRN-type counterparts (FIG. 4c). Combination therapy with TRAIL and Lorlatinib is therefore effective to heterogeneous neuroblastoma populations in vitro.

The results of clinical trials with TRAIL have not led to inclusion of this compound in regular treatment modalities. The inventors therefore tested the effect of TRAIL in a mouse xenograft model. The inventors first tested TRAIL in the neuroblastoma cell line SK-N-AS which has a MES phenotype and forms predominantly MES-type tumours after xenografting. The inventors generated SK-N-AS cells with an inducible recombinant TRAIL transgene. Doxycycline treatment induces strong TRAIL expression on their cell membranes and results in apoptosis of a large proportion of the cells in vitro (FIG. 7b-d). The inventors tested whether this proceeds via activation of membrane-expressed TRAIL receptors. We co-cultured the SK-N-AS-TRAIL cells with either GFP-labelled IMR32 or SH-EP2 neuroblastoma cells in a 3D model. Doxycycline-induced TRAIL expression killed the MES-type SH-EP2 cells but not the ADRN-type IMR32 cells, confirming the specific killing of MES cells via juxtacrine signalling (FIG. 4d). Xenografting of the SK-N-AS-TRAIL cells in mice resulted in rapidly growing tumours which maintained their MES phenotype as shown by immunohistochemical (IHC) and mRNA profiling analysis (FIGS. 7e and 4f). Induction of TRAIL by doxycycline treatment of the established tumours (200 mm³) induced a complete regression, which was maintained during the five-week doxycycline stimulation (FIG. 4e). TRAIL is therefore effective to MES-type tumour cells in vivo.

Most human neuroblastoma tumours include at diagnosis only a low percentage of MES-type cells, which were proposed to preferentially survive therapy and may play a role in relapse development⁵. The inventors therefore tested whether eradication of MES cells in a predominantly ADRN-type neuroblastoma would delay relapse development. SH-SY5Y cells have an ADRN phenotype in vitro and form rapidly growing xenografts mainly consisting of ADRN cells (FIGS. 8g and 8h). A 4-week treatment with TRAIL alone did not inhibit tumour growth and even induced a slight acceleration (FIG. 4f). A six-week treatment with Lorlatinib alone induced rapid regression of the tumours to marginally palpable rests. After cessation of Lorlatinib treatment, about half of the tumours relapsed after 7 to 16 days, which confirms a previous report¹². Combined treatment with Lorlatinib and TRAIL also induced rapid tumour regression, followed by relapses in about half of the mice. However, the addition of TRAIL delayed tumour outgrowth with about two weeks, as evident by a significant (p<0.001; ANOVA) reduction of the average tumour volume at day 67 (FIG. 4f). These data show that targeting of the minor population of MES cells in neuroblastoma can abate tumour regrowth.

### Material and Methods

### Cell Culture

Patient-derived neuroblastoma cell lines (691, 717T) were derived and cultured in neural stem cell (NSC) medium as described^{5,25}. Serum-cultured cell lines were maintained as described earlier²⁶. Early-passage parental NBL-W cells showed heterogeneous growth of floating spheres and attached cells with lamellipodia²⁷. NBLW-MES and NBLW-ADRN were singled out from the parental NBLW by culture conditions (the MES and ADRN phenotype differ in attachment strength to culture plates). Cell line authenticity was verified using Short Tandem Repeat (STR) analysis. Cells were routinely checked for the presence of mycoplasma using MycoAlert detection kit (Lonza).

### Generation of Transgenic Cell Lines

The SK-N-AS cell line with inducible TRAIL expression was generated by cloning human TRAIL from pUNO1-hTRAILa vector (InvivoGen) into the lentiviral pINDUCER (pIND) system (Invitrogen). Expression of the TRAIL transgene was induced by the addition of doxycycline to the culture medium at a final concentration of 300 ng/ml. SH-SY5Y-GFP, IMR-GFP and SH-EP2-GFP were generated by lentiviral (pLenti, Invitrogen) transduction of turboGFP (SHC003, Sigma) and SH-EP2-RFP by lentiviral (pLenti, invitrogen) transduction of near-infrared fluorescent protein NirFP (Evrogen). Expression of the NOTCH3-IC transgene was induced by the addition of doxycycline to the culture medium at a final concentration of 100 ng/ml.

### Gene Expression Profiling and Analysis of Micro-Array Data

Total RNA from NBLW-ADRN and -MES cells in vitro, SK-N-AS-TRAIL and SH-SY5Y tumours was harvested and isolated using Trizol reagent (Invitrogen) as described⁵. RNA quality was verified on a BioAnalyzer (Agilent). RNA was hybridized on Affymetrix HG U133A plus2.0 gene chips and scanned data were normalized using the MASS5.0 algorithm.

Two groups of cell lines, MES (SK-N-AS, SH-EP2, GI-MEN, NBLW-MES, 691-MES, 717T-MES, 700B-MES and ACN) and ADRN (SKNFI, IMR32, SJNB10, SJNB1, AMC106, NBL-W, NBLW-ADRN, CHP134, SK-N-BE, N206, 691-ADRN, NMB, CHP134, SK-N-SH, NGP-04, KCNR, TR-14, LANS, LAN1, SH-SY5Y, SJNB6, 700T, 711T, 717T-ADRN, UHGNP, 753T, 772T and 700B-ADRN) were generated with the custom track function. Differentially expressed genes of the Gene Ontology gene set 97190 (apoptosis pathway; 588 genes) between these groups of cell lines were identified by ANOVA on 2log-transformed gene expression values using a p value cut-off of p<0.01 and a FDR correction for multiple testing. A minimum of expression of 50 was required. This analysis identified 90 MES/ADRN-differentially expressed apoptosis genes (Table 1).

### ChIP-Sequencing Analysis

ChIP-sequencing profiles for H3K27ac in MES- and ADRN cell lines are available from GEO (GSE90805). H3K27ac ChIP-sequencing data from SH-SY5Y-TetR-NOTCH3-IC cells are available from GEO (GSE116893) and were analysed as described5. Enhancers (H3K27ac) were determined by ChIP-sequencing in NBLW-ADRN and -MES cell lines. ChIP-sequencing using H3K27ac antibody (4729, Abcam) was performed as described5 and is available from GEO (GSEnummer). ChIP-seq data was processed and visualized on the R2 genomics analysis and visualization platform (http://r2.amc.nl/) as described⁵.

### Western Blot Analysis

Western blotting was performed according to standard protocols. Total cell lysates were made in RIPA-buffer. Protein was transferred to nitrocellulose membrane (RPN203D, GE Healthcare). Membranes were blocked for 1 hour at RT, incubated at 4° C. overnight or 2 hours at RT with primary antibody and incubated for 1 hour at RT with secondary antibodies in either 2% PBA (RPN418, GE Healthcare), 5% BSA (10735086001, Sigma-Aldrich) or OBB (829-31080, LI-COR) in PBS 0.1% TWEEN (P1379, Sigma).

Primary antibodies ALK (3633), DLK1 (2069), CASP8 (9746), NOTCH3 (5276), HES1 (11988), DBH (8586), VIM (5741), SNAI2 (9585), YAP/TAZ (8414) were obtained from Cell Signaling. Other primary antibodies include FN1 (AF1918, R&D systems), PHOX2A (sc81978), PHOX2B (sc376997), TH (sc25269, Santa Cruz), PRRX1 (HPA-051084, Sigma) and β-actin (ab6276, Abcam).

Secondary antibodies used for chemiluminescent detection were donkey anti-rabbit-HRP (NA 9340V, GE healthcare), donkey anti-sheep/goat-HRP (STAR88P, Biorad) or sheep anti-mouse-HRP (NXA931, GE healthcare). Chemiluminescent detection was done using the ECL Prime Western Blotting kit (RPN2232, GE healthcare) and developed on a ImageQuant LAS 4000 (28-9558-10, GE healthcare). For infrared fluorescent detection, membranes were incubated with secondary antibody Goat anti-mouse-IRDye® 680RD (926-68070, LI-COR) and scanned on an Odyssey Infrared imaging System, (LIC-9201-00, LI-COR).

### Cell Viability Assays

Cells were plated in 96-well plates, one day before the addition of Lorlatinib (HY-12215, MedChemExpress) and/or TRAIL (Peprotech) to the culture medium. Seeding densities were optimized for each cell line so that the control, non-treated cells would reach 70-80% confluence by the end of the experiment. Lorlatinib and TRAIL were added for 96 or 120 hours. Metabolic activity was measured by 3-(4,5-dimethylthiazol-2-yl)-2,5-diphenyltetrazolium bromide (MTT) assay, according to the manufacturer's instructions with minor modifications. Sextuple or triplicate measurements were used for each experimental condition. Two-sided t-tests assuming equal variance were used to determine significance. Experiments were repeated at least twice.

### FACS Analysis

Cell membrane expression of ALK in SH-SY5Y-NOTCH3IC and TRAIL in SK-N-AS-TRAIL was measured after 24 hours doxycycline treatment. Attached cells were collected, spun down and washed twice with FACS buffer (PBS supplemented with 0.5% Bovine Serum Albumin (Sigma) and 4 mM EDTA). 1×10⁶ cells in 100-200 μl FACS buffer was supplemented with 1:50 ALK Alexa Fluor 647 (sc-398791, Santa Cruz) or 1:50 Alexa Fluor 647 Mouse IgG2a, kappa Isotype control (400234, ITK Diagnostics) and incubated for 1 hour at 4° C. in the dark. For TRAIL detection, 1:50 TRAIL antibody (3219, Cell Signaling) in FACS buffer was added, incubated for 1 hour at 4° C., washed once with FACS buffer and incubated for 30 min at 4° C. in the dark with 1:5000 secondary antibody Alexa Fluor 647 (A31573, Life technologies). After incubation cells were spun down, washed twice with FACS buffer to remove not bound antibodies and analysed using flow cytometer (BD biosciences) and BD Accuri C6 software or Flowing Software 2.5.1.

GFP and RFP labelled cells were plated at given densities (see Figure description), collected by trypsin treatment, washed twice with FACS buffer and analysed using flow cytometer (BD biosciences) and BD Accuri C6 software.

### Spheroid Assays

Spheroid assays were performed as described²⁸. A suspension of 10000 cells of 3:1 SKNAS-TRAIL:SH-EP2-GFP or 3:1 SKNAS-TRAIL:IMR32-GFP was added in triplicate to 96 well round bottom suspension plates. After 48 hours incubation with 0 or 300 ng/ml doxycycline bright-field and fluorescent images of the 3D cultures were taken.

### Production and Purification of TRAIL for In Vivo Experiments

Recombinant His-tagged soluble human TRAIL containing pQE-hTR plasmid (Addgene #21811)29 was expressed in BL21-CodonPlus (DE3)-RIPL Competent Cells (Agilent). For the detailed description of production, isolation and purification see Extended data Material and Methods.

### In Vivo Tumorigenicity and Histological Analysis

For tumour growth assays, 5×10⁶ SH-SY5Y cells were suspended in 200 μl of a 50% Matrigel (BD, 354234) solution in PBS and subcutaneously injected in NMRI-Foxn1nu/nu mice (NKI; Janvier). After tumour outgrowth to around 200-250 mm³, mice were randomized to control or treatment groups. The control group received 150 μl PBS daily intraperitoneally (I.P.) for 4 weeks (day 1-day 28). The TRAIL group received 150 μg/day through I.P. for 4 weeks (day 1-day 28). The Lorlatinib group received 5 mg/kg twice a day through oral gavage for 6 weeks (day 1-day 42) and 4 weeks (day 29-day 56) of PBS (150 μl/day I.P.). The combination group received Lorlatinib twice a day for 6 weeks (day 1-day 42) and 4 weeks TRAIL (day 29-day 56). Tumour size was measured three times a week. Mice were sacrificed at the humane endpoint (tumor size >1500 mm³), the tumours were isolated and divided in two parts. One tumour piece was fresh frozen in liquid nitrogen and RNA was isolated using Trizol reagent. The other tumour piece was fixed in 4% (w/v) buffered formaldehyde (Klinipath) and embedded in paraffin for histological analyses. In vivo experiments were conducted at the NKI therapeutical intervention unit after ethical approval from the animal experiments committee of the NKI was obtained (AvD:30100 2015 407 appendix 1; WP8156).

For the SK-N-AS-TRAIL xenograft experiments, 1×10⁶ cells were suspended in 200 μl of a 50% Matrigel solution in PBS and subcutaneously injected in NMRI-Foxn1nu/nu mice. After tumour outgrowth to around 200-250 mm³, mice were randomized to control or treatment groups. The DOX group was provided drinking water with 200 μg/ml doxycycline. DOX-containing water was refreshed weekly. Control mice received regular drinking water ad lib. Mice were screened regularly to follow tumour formation and were sacrificed before tumour reached 1500 mm³. All animal experiments were conducted under the institutional guidelines and according to the law and approved in DAG155 by the AMC animal ethics committee.

### Immunohistochemistry

Sections (4 μm) from formalin-fixed, paraffin-embedded SH-SY5Y-NOTCH3IC tumours were analysed by standard IHC protocols³⁰. The primary antibody to ALK (3336 from Cell Signaling) was used 1:500 after Tris-EDTA pH 9.0 treatment. The rabbit primary antibody was detected using biotin-free species-specific HRP-conjugated polymers (BrightVision anti-Rb IgG/HRP, Immunologic). HRP activity was developed with DAB+ (K3468, Dako).

### Extended Data: Materials and Methods

### Production and Purification of Recombinant His-Tagged Soluble Human TRAIL

Recombinant His-tagged soluble human TRAIL containing pQE-hTR plasmid (Addgene, #21811) was expressed in BL21-CodonPlus (DE3)-RIPL Competent Cells (Agilent). A single colony of recombinant cells was picked from a streaked LB agar plate supplemented with 200 μg/ml ampicillin and 45 μg/ml chloramphenicol and inoculated in 30 ml of LB broth supplemented with 200 μg/ml ampicillin and 45 μg/ml chloramphenicol in a 250 ml flask. The culture was grown overnight (˜16 h) in a 37° C. shaking incubator. The overnight seed culture was diluted to an OD₆₀₀=0.1 in super broth (32 g/L tryptone, 20 g/L yeast extract, 5 g/L NaCl) with 100 μg/ml ampicillin and 45 μg/ml chloramphenicol and incubated at 37° C. in a shaking incubator to reach an OD₆₀₀=0.6. 1 mM Isopropyl-1-thio-b-D-galactopyranoside (IPTG, Sigma) was added to induce recombinant protein expression, and bacterial cells were incubated for another 4 h at 37° C. in a shaking incubator. Cells were spun down and resuspended in 5 ml B-PERT™ Complete Bacterial Protein Extraction Reagent (Sigma-Aldrich) per gram cells supplemented with 1 tablet/50 ml complete EDTA-free protease inhibitor (Sigma-Aldrich). Cell suspension was homogenized in a tissue grinder followed by sonication in ice and spun down to collect the supernatant. Supernatant was adjusted to pH 8 with 0.5M Na2HPO4 (pH 8). HisPur™ Ni-NTA Chromatography Cartridges (Thermo Fisher) were used to bind recombinant His-tagged soluble human TRAIL in an NGC Chromatography system (Bio-rad). After washing with buffer A (50 mM Na2HPO4 pH 8, 500 mM Na Cl, and 20 mM imidazole) and buffer A supplemented with 0.1% Triton X-114, recombinant His-tagged soluble human TRAIL was eluted with elution buffer (50 mM Na2HPO4 pH 8, 500 mM NaCl, and 300 mM imidazole) and redirected to Bio-Scale™ Mini Bio-Gel® P-6 Desalting Cartridges (Bio-rad) to exchange elution buffer for PBS. The purity of the eluted protein was confirmed by SDS-PAGE and Coomassie Blue gel staining and endotoxin levels were quantified with Pierce LAL chromogenic Endotoxin Quantitation kit (Thermo Fisher). The concentration of the protein was checked using a Bio-Rad protein assay.

### Sequence Analysis

ALK was sequenced using Biometra (Biosciences).

### Cell Count Assays

To determine the effect of doxycycline-induced TRAIL expression on SK-N-AS cells, 150.000 SK-N-AS-TRAIL cells were seeded in triplicate in 6 well/plates in 2 ml medium supplemented with or without 300 ng/ml doxycycline. Cell number was determined using a coulter counter (Beckman) at every 3rd or 4th day and cells were reseeded at similar cell number as at the start until the end of the experiment. The proliferation factor was defined as the ratio of the cells counted and the cells plated.

### FACS Analysis Apoptosis

Sub-G1 fractions were determined after 72 hours treatment with 300 ng/ml doxycycline. Both the attached and the floating SK-N-AS-TRAIL cells were fixed with 100% ethanol at −20 ° C. After fixing, the cells were stained with 0.05 mg/ml propidium iodide and 0.05 mg/ml RNAse A in PBS. After 1 hour incubation, DNA content of the nuclei was analysed using a Accuri C6 flow cytometer (BD biosciences). The cell cycle distribution and apoptotic sub G1 fraction was determined using BD Accuri C6 software.

### Immunohistochemistry

Sections (4 μm) from formalin-fixed, paraffin-embedded SH-SY5Y and SK-N-AS-TRAIL tumours were analysed similar as for as described above. The primary antibodies used were to ALK (3336 from Cell Signaling, Tris-EDTA pH 9.0, 1:500), DBH (8586 from Cell Signaling, Tris-EDTA pH 9.0, 1:100, CASP8 (PA5-32294 from Thermo Scientific, citrate pH 6.0, 1:1000), YAP/TAZ (8418 from Cell Signaling, citrate pH 6.0, 1:200) and PDGFRβ (4564 from Cell Signaling, Tris-EDTA pH 9.0, 1:500).

TABLE 1
Differentially Expressed genes of the apoptosis pathway
(GO 97190 geneset; n = 588) between mesenchymal and
adrenergic neuroblastoma celllines (n = 90).

| Gene (HUGO) | R-value | p-value | sublist |
|---|---|---|---|
| P4HB | 0.811 | 4.91E+07 | MES >= ADRN |
| GSN | 0.797 | 6.97E+07 | MES >= ADRN |
| PEA15 | 0.647 | 2.86E−04 | MES >= ADRN |
| BCLAF1 | −0.633 | 4.48E−04 | MES < ADRN |
| DAP | 0.644 | 3.05E−04 | MES >= ADRN |
| TMEM109 | 0.677 | 1.04E−04 | MES >= ADRN |
| CUL3 | −0.542 | 3.86E−03 | MES < ADRN |
| SFPQ | −0.524 | 5.74E−03 | MES < ADRN |
| KRT18 | 0.544 | 3.70E−03 | MES >= ADRN |
| CIB1 | 0.707 | 4.18E+08 | MES >= ADRN |
| ITGAV | 0.594 | 1.19E−03 | MES >= ADRN |
| HTT | −0.726 | 2.09E+09 | MES < ADRN |
| PRKCD | 0.592 | 1.21E−03 | MES >= ADRN |
| SERPINE1 | 0.648 | 2.78E−04 | MES >= ADRN |
| TNFAIP3 | 0.576 | 1.80E−03 | MES >= ADRN |
| ATF3 | −0.524 | 5.68E−03 | MES < ADRN |
| SP100 | 0.625 | 5.15E−04 | MES >= ADRN |
| MSH6 | −0.669 | 1.42E−04 | MES < ADRN |
| SIAH1 | −0.547 | 3.40E−03 | MES < ADRN |
| ARRB2 | −0.612 | 7.15E−04 | MES < ADRN |
| LMNA | 0.787 | 9.71E+07 | MES >= ADRN |
| STX4 | 0.623 | 5.38E−04 | MES >= ADRN |
| FAIM2 | −0.701 | 5.16E+09 | MES < ADRN |
| BCL2 | −0.504 | 9.28E−03 | MES < ADRN |
| ACVR1 | 0.613 | 7.22E−04 | MES >= ADRN |
| P2RX4 | 0.571 | 2.04E−03 | MES >= ADRN |
| IKBKE | 0.689 | 7.70E+09 | MES >= ADRN |
| FAS | 0.716 | 2.90E+09 | MES >= ADRN |
| E2F1 | −0.554 | 2.95E−03 | MES < ADRN |
| SRPX | 0.788 | 1.13E+08 | MES >= ADRN |
| TLR3 | 0.697 | 5.55E+08 | MES >= ADRN |
| DBH | −0.625 | 5.08E−04 | MES < ADRN |
| LY96 | 0.594 | 1.19E−03 | MES >= ADRN |
| TNFRSF1A | 0.685 | 8.15E+09 | MES >= ADRN |
| PDIA3 | 0.554 | 2.95E−03 | MES >= ADRN |
| HMGB2 | −0.668 | 1.42E−04 | MES < ADRN |
| PAK2 | −0.555 | 2.96E−03 | MES < ADRN |
| LGALS3 | 0.742 | 1.04E+09 | MES >= ADRN |
| IFI16 | 0.654 | 2.30E−04 | MES >= ADRN |
| KRT8 | 0.530 | 4.98E−03 | MES >= ADRN |
| PRKRA | −0.603 | 9.68E−04 | MES < ADRN |
| NUPR1 | 0.735 | 1.44E+09 | MES >= ADRN |
| CASP4 | 0.759 | 4.35E+08 | MES >= ADRN |
| MSH2 | −0.536 | 4.37E−03 | MES < ADRN |
| ARHGEF2 | 0.587 | 1.36E−03 | MES >= ADRN |
| CRADD | −0.541 | 3.88E−03 | MES < ADRN |
| CTNNA1 | 0.569 | 2.06E−03 | MES >= ADRN |
| MLLT11 | −0.592 | 1.22E−03 | MES < ADRN |
| BMP4 | 0.571 | 2.04E−03 | MES >= ADRN |
| CD44 | 0.685 | 8.32E+09 | MES >= ADRN |
| CAV1 | 0.682 | 8.90E+09 | MES >= ADRN |
| FEM1B | −0.559 | 2.64E−03 | MES < ADRN |
| TCF7L2 | 0.577 | 1.79E−03 | MES >= ADRN |
| NACC2 | 0.562 | 2.47E−03 | MES >= ADRN |
| MAPK8IP1 | −0.622 | 5.44E−04 | MES < ADRN |
| PHIP | −0.594 | 1.21E−03 | MES < ADRN |
| SNAI2 | 0.540 | 3.96E−03 | MES >= ADRN |
| ACVR1B | −0.777 | 1.53E+08 | MES < ADRN |
| DIDO1 | −0.565 | 2.29E−03 | MES < ADRN |
| CASP8 | 0.806 | 4.78E+07 | MES >= ADRN |
| FASTK | 0.580 | 1.71E−03 | MES >= ADRN |
| EYA1 | −0.525 | 5.63E−03 | MES < ADRN |
| PARP2 | −0.557 | 2.83E−03 | MES < ADRN |
| CD24 | −0.723 | 2.30E+09 | MES < ADRN |
| BAG3 | 0.631 | 4.68E−04 | MES >= ADRN |
| TNFRSF12A | 0.833 | 1.38E+07 | MES >= ADRN |
| PHLDA3 | 0.602 | 9.66E−04 | MES >= ADRN |
| TFPT | 0.548 | 3.38E−03 | MES >= ADRN |
| AEN | −0.598 | 1.09E−03 | MES < ADRN |
| ZMAT4 | −0.702 | 5.16E+09 | MES <ADRN |
| SERINC3 | 0.617 | 6.38E−04 | MES >= ADRN |
| RBCK1 | 0.540 | 3.92E−03 | MES >= ADRN |
| CASP8AP2 | −0.627 | 5.15E−04 | MES < ADRN |
| FIGNL1 | −0.633 | 4.40E−04 | MES < ADRN |
| BRSK2 | −0.786 | 8.98E+07 | MES < ADRN |
| FOXO3 | −0.578 | 1.76E−03 | MES < ADRN |
| YAP1 | 0.700 | 5.11E+09 | MES >= ADRN |
| DAB2IP | −0.512 | 7.68E−03 | MES < ADRN |
| DNAJC10 | 0.680 | 9.30E+09 | MES >= ADRN |
| UBE2K | −0.650 | 2.69E−04 | MES < ADRN |
| DYNLL2 | −0.571 | 2.02E−03 | MES < ADRN |
| MAPK8 | −0.626 | 5.22E−04 | MES < ADRN |
| DNM1L | −0.577 | 1.78E−03 | M ES < ADRN |
| GSK3B | −0.627 | 5.10E−04 | MES < ADRN |
| IL6R | 0.718 | 2.83E+09 | MES >= ADRN |
| SENP1 | −0.551 | 3.13E−03 | MES < ADRN |
| E2F2 | −0.655 | 2.27E−04 | MES < ADRN |
| BCL2L12 | 0.688 | 7.49E+09 | MES >= ADRN |
| PPP1R15A | 0.500 | 9.94E−03 | MES >= ADRN |
| PPM1F | 0.591 | 1.22E−03 | MES >= ADRN |

The experiment described above was repeated using the ALK inhibitor Alectinib (Roche) instead of Lorlatinib. FIG. 8 shows preferential killing of ADRN-type neuroblastoma cells, and resistance of MES-type neuroblastoma cells.

### REFERENCES

1 Tirosh, I. et al. Dissecting the multicellular ecosystem of metastatic melanoma by single-cell RNA-seq. Science 352, 189-196, doi:10.1126/science.aad0501 (2016).

2 Tirosh, I. et al. Single-cell RNA-seq supports a developmental hierarchy in human oligodendroglioma. Nature 539, 309-313, doi:10.1038/nature20123 (2016).

3 Patel, A. P. et al. Single-cell RNA-seq highlights intratumoral heterogeneity in primary glioblastoma. Science 344, 1396-1401, doi:10.1126/science.1254257 (2014).

4 Suva, M. L. et al. Reconstructing and reprogramming the tumor-propagating potential of glioblastoma stem-like cells. Cell 157, 580-594, doi:10.1016/j.cell.2014.02.030 (2014).

5 van Groningen, T. et al. Neuroblastoma is composed of two super-enhancer-associated differentiation states. Nat Genet 49, 1261-1266, doi:10.1038/ng.3899 (2017).

6 Boeva, V. et al. Heterogeneity of neuroblastoma cell identity defined by transcriptional circuitries. Nat Genet 49, 1408-1413, doi:10.1038/ng.3921 (2017).

7 Maris, J. M. Recent advances in neuroblastoma. N Engl J Med 362, 2202-2211, doi:10.1056/NEJMra0804577 (2010).

8 Mosse, Y. P. et al. Identification of ALK as a major familial neuroblastoma predisposition gene. Nature 455, 930-935, doi:10.1038/nature07261 (2008).

9 Chen, Y. et al. Oncogenic mutations of ALK kinase in neuroblastoma. Nature 455, 971-974, doi:10.1038/nature07399 (2008).

10 Janoueix-Lerosey, I. et al. Somatic and germline activating mutations of the ALK kinase receptor in neuroblastoma. Nature 455, 967-970, doi:10.1038/nature07398 (2008).

11 George, R. E. et al. Activating mutations in ALK provide a therapeutic target in neuroblastoma. Nature 455, 975-978, doi:10.1038/nature07397 (2008).

12 Infarinato, N. R. et al. The ALK/ROS1 Inhibitor PF-06463922 Overcomes Primary Resistance to Crizotinib in ALK-Driven Neuroblastoma. Cancer Discov 6, 96-107, doi:10.1158/2159-8290.CD-15-1056 (2016).

13 Schulte, J. H. et al. MYCN and ALKF1174L are sufficient to drive neuroblastoma development from neural crest progenitor cells. Oncogene 32, 1059-1065, doi:10.1038/onc.2012.106 (2013).

14 Berry, T. et al. The ALK(F1174L) mutation potentiates the oncogenic activity of MYCN in neuroblastoma. Cancer Cell 22, 117-130, doi:10.1016/j.ccr.2012.06.001 (2012).

15 van Groningen, T. et al. A NOTCH feed-forward loop drives reprogramming from adrenergic-to mesenchymal state in neuroblastoma. (submitted; in revision).

16 ClinicalTrials.gov.; NCT03107988, NCT01742286, NCT02780128, NCT03213652, NCT03155620, NCT03126916, NCT00939770, NCT01121588.

17 Ross, R. A., Spengler, B. A. & Biedler, J. L. Coordinate morphological and biochemical interconversion of human neuroblastoma cells. J Natl Cancer Inst 71, 741-747 (1983).

18 Debruyne, D. N. et al. ALK inhibitor resistance in ALK(F1174L)-driven neuroblastoma is associated with AXL activation and induction of EMT. Oncogene 35, 3681-3691, doi:10.1038/onc.2015.434 (2016).

19 Hoek, K. S. et al. In vivo switching of human melanoma cells between proliferative and invasive states. Cancer Res 68, 650-656, doi:10.1158/0008-5472.CAN-07-2491 (2008).

20 Lim, J. S. et al. Intratumoural heterogeneity generated by Notch signalling promotes small-cell lung cancer. Nature 545, 360-364, doi:10.1038/nature22323 (2017).

21 Liau, B. B. et al. Adaptive Chromatin Remodeling Drives Glioblastoma Stem Cell Plasticity and Drug Tolerance. Cell Stem Cell 20, 233-246 e237, doi:10.1016/j.stem.2016.11.003 (2017).

22 Tamura, K. et al. Expansion of CD133-positive glioma cells in recurrent de novo glioblastomas after radiotherapy and chemotherapy. J Neurosurg 119, 1145-1155, doi:10.3171/2013.7.JNS122417 (2013).

23 Boshuizen, J. et al. Cooperative targeting of melanoma heterogeneity with an AXL antibody-drug conjugate and BRAF/MEK inhibitors. Nat Med 24, 203-212, doi:10.1038/nm.4472 (2018).

24 Rambow, F. et al. Toward Minimal Residual Disease-Directed Therapy in Melanoma. Cell 174, 843-855 e819, doi:10.1016/j.cell.2018.06.025 (2018).

25 Biehs, B. et al. A cell identity switch allows residual BCC to survive Hedgehog pathway inhibition. Nature 562, 429-433, doi:10.1038/s41586-018-0596-y (2018).

26 Sanchez-Danes, A. et al. A slow-cycling LGR5 tumour population mediates basal cell carcinoma relapse after therapy. Nature 562, 434-438, doi:10.1038/s41586-018-0603-3 (2018).

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or embodiments described herein. It is intended that the appended claims be interpreted to include all such equivalents.

1. A pharmaceutical product comprising therapeutically effective amounts of:
(a) an ALK inhibitor or an antibody-drug conjugate directed to the ALK receptor, and
(b) a TNF-related apoptosis-inducing ligand (TRAIL) receptor agonist.

2. The pharmaceutical product according to claim 1 wherein said ALK inhibitor is Lorlatinib or Alectinib.

3. The method of claim 4, wherein said ALK-positive neoplasm is neuroblastoma.

4. A method of treating an ALK-positive neoplasm in a patient in need thereof, the method comprising administering to the patient the pharmaceutical product of claim 1.

5. The method of claim 4, wherein said an ALK-positive neoplasm is selected from the group consisting of anaplastic large cell lymphoma, non-small-cell lung cancer, inflammatory myofibroblastic tumor, neuroblastoma and colorectal cancer.

6. The method of claim 4, wherein said ALK-positive neoplasm has at least one oncogenic mutation in the ALK gene.

7. The method of claim 4, wherein said ALK-positive neoplasm is characterized by the presence of malignant cells lacking ALK expression.

8. The method of claim 4, wherein said ALK-positive neoplasm is resistant to at least one ALK inhibitor or to an antibody-drug conjugate directed to the ALK receptor.
